(19) United States
(12) Patent Application Publication
(10) Pub. No.: US 2006/0247154 A1
(43) Pub. Date: Nov. 2, 2006

(54) Concanavalin a, methods of expressing, purifying and characterizing concanavalina, and sensors including the same

(75) Inventors: Stephen J. Palmieri, Worcester, MA (US); Dylan A. Bulseco, Princeton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: LifeScan, Inc., Milpitas, CA (US)

(21) Appl. No.: 11/363,373

(22) Filed: Feb. 24, 2006

Related U.S. Application Data
(60) Provisional application No. 60/655,756, filed on Feb. 24, 2005.

(51) Int. Cl. A61K 38/16; C07K 14/42

(52) U.S. Cl. 514/8; 530/370

(57) ABSTRACT

A novel method for purifying various lectins is disclosed. More specifically a novel method for purifying Concanavalin A is set forth. Methods of expressing purifying and characterizing a mutant Concanavalin A, and sensors including the foregoing are also disclosed.

### CROSS REFERENCE TO RELATED APPLICATION

This application claims benefit of priority to U.S. Provisional Patent Application Ser. No. 60/655,756, filed on Feb. 24, 2005, the entirety of which is incorporated by reference.

### FIELD OF THE INVENTION

This invention relates to methods of expressing, purifying and characterizing lectins generally and Concanavalin A and mutants thereof, specifically. The instant invention also includes sensors incorporating purified Concanavalin A and mutants thereof.

### BACKGROUND OF THE INVENTION

Lectins are a family of carbohydrate binding proteins found both in prokaryotes and eukaryotes including: classical lectins, which are plant derived; and carbohydrate binding proteins derived from animals. Studies have identified the structure of lectin genes in soybean, French bean and pea, as well as other sources. Concanavalin A (ConA), a seed lectin of the Canavalia species is synthesized via a somewhat unique mechanism in the maturing seeds. The precursor to ConA, a glycosylated protein, undergoes post-translational modification including the ligation of cleaved polypeptides. This process is required for the production of active protein.

ConA, itself, is a family of tetrameric plant lectin composed of four 26 kDa monomeric subunits that recognize and bind to carbohydrates. Purified ConA from jack beans (Canavalia ensiformis) is commonly used as a molecular probe for the investigation of glycoproteins. It specifically binds D-mannose and D-glucose with high affinity, and also binds other proteins independent of glycosylation state.

As mentioned above, ConA is initially synthesized as a precursor protein (pre-pro ConA) that undergoes multiple post-translational modifications required for activation (Sheldon, et al., 1996; Brennan, et. al., 1993; Sheldon, et. al., 1992; Carrington, et. al., 1985). In the plant, these modifications include removal of the signal peptide, deglycosylation, proteolytic cleavage, transposition and re-ligation (transpeptidation) of the N- and C-terminal halves to generate the mature 26 kDa ConA monomer (FIGS. 1 and 2). Monomeric ConA assembles into tetramers through a dimer intermediate in a pH dependent manner. Analyses of commercially available sources of ConA purified from Jack bean meal reveal the presence of other contaminating protein bands (14 kDa and 12 kDa) as determined by SDS-PAGE, presumably resulting from incomplete ligation of the processed peptide fragments (FIG. 2). The incompletely processed fragments are still capable of assembling into functional tetramers with other fragments or with full-length monomers. As a result purified commercial natural ConA tetramers include both full length and fragmented ConA monomers.

ConA's ability to specifically bind D-mannose and D-glucose with high-affinity has made it useful as a tool for determining the blood and tissue glucose levels in patients with diabetes. In particular, ConA has been thought to be useful in the design and manufacture of devices for the measurement of glucose in biological fluids, particularly blood. Producing ConA in commercial quantities with sufficient purity to be useable as a component in such a device would be very useful.

### SUMMARY OF THE INVENTION

The invention provides for compositions comprising purified polypeptides, more specifically purified lectins, specifically purified Concanavalin A. The invention also provides an improved method of producing recombinant lectins in general and Concanavalin A in particular. In addition, the invention provides for novel polypeptides and nucleic acids encoding those polypeptides, in particular, novel mutant Concanavalin A. The invention also provides for sensors comprising the polypeptides of the invention.

More specifically, the invention provides for a composition comprising a substantially purified lectin of interest wherein the composition is at least 95% pure. In one aspect of the invention, the composition comprises a lectin polypeptide, wherein the lectin comprises greater than 95% by weight of the total protein of the composition. In yet another aspect, the composition comprises a lectin polypeptide, wherein the composition has a purity greater than 95% as determined by relative peak area integration. In a further embodiment the composition has a purity greater than 97% as determined by relative peak area integration. In still another aspect of the invention, the lectin polypeptide comprises recombinant Concanavalin A. The invention also provides for a composition wherein the lectin is a tetramer, dimer, or monomer. In another embodiment the lectin polypeptide comprises a mutant recombinant Concanavalin A polypeptide, specifically, the polypeptide of SEQ ID NO:15.

The invention also provides a method of producing a recombinant lectin of interest, comprising inducing expression of said lectin in the bacterial culture; lysing the cells of the bacterial culture to produce an inclusion body fraction; purifying the inclusion body fraction; solulibilizing the inclusion bodies in the inclusion body fraction so that the lectin of interest is present in solution; denaturing the lectin of interest; allowing the lectin of interest to refold in solution; and purifying the resulting solution.

In one embodiment of the production method, the cells of the bacterial culture have been transformed by a vector comprising a kanamycin resistance gene. In some aspects of the method of the invention, the bacterial cell culture is induced with IPTG in the absence of kanamycin. In yet another embodiment denaturing the lectin of interest occurs at a pH of less than 5.

In some embodiments of the production method, the solution is purified by affinity chromatography. In other embodiments of the method, the solution is purified by size-exclusion chromatography. In yet another embodiment of the production method, the solution is purified by both size-exclusion chromatography and affinity chromatography. In another embodiment of the invention, the lectin is a member of a family of proteins that specifically binds at least one of glucose and mannose. In another embodiment of the production method of the invention, the lectin is a form of Concanavalin A. In still another embodiment, the Concanavalin A is a mutant form of the Concanavalin A polypeptide of SEQ ID NO:15.

The invention also provides for a method of purifying a lectin of interest comprising adding a denaturing, chaotropic agent to a solution of the lectin having a pH less than 5, and subjecting said solution to size exclusion chromatography. In one embodiment, the lectin of interest is Concanavalin A.

The invention further provides for a composition comprising a substantially purified lectin having less than about 150 ng of Host Cell Protein (HCP) per mg of purified lectin. In one aspect of the invention the lectin of the composition is Concanavalin A. In still another aspect, the Concanavalin A is a mutant form of Concanavalin A, specifically, the polypeptide of SEQ ID NO:15.

The invention also provides for an isolated nucleic acid sequence encoding a mutant form of a natural Concanavalin A. In one embodiment, the isolated nucleic acid of the invention specifically comprises SEQ ID NO:16.

The invention also contemplates the isolated nucleic acid encoding a mutant Concanavalin A operatively linked to a promoter. The invention further provides a host cell that contains the nucleic acid of the invention, operatively linked to a promoter and expressing the encoded protein. In one embodiment of the invention the isolated nucleic acid sequences of the invention encodes the polypeptide of SEQ ID NO:15.

The invention also provides for a method of producing a Concanavalin A exhibiting reduced precipitation during purification, comprising performing a mutation to the nucleic acid sequence of a Concanavalin A wherein the mutation encodes for an amino acid change, the amino acid change converting an acidic amino acid to a neutral amino acid. The invention also provides a method using a vector comprising an inducible promoter, a kanamycin resistance gene and a nucleic acid sequence encoding for a form of Concanavalin A. In one aspect of the invention, the nucleic acid sequence of the vector is comprised of the sequence of SEQ ID NO:16.

The invention further provides for sensors comprising a mutant form of Concanavalin A. In one aspect of the invention, the mutant form of Concanavalin A has at least one mutation encoding for an amino acid change, the amino acid change converting an acidic amino acid to a neutral amino acid. In a further embodiment of the sensors of the invention, the mutant Concanavalin A comprises the polypeptide of SEQ ID NO:15.

The invention also provides for sensors comprising a donor, and an acceptor, wherein the mutant Concanavalin A is labeled with at least one of the donor and the acceptor. In one embodiment the sensor comprises a fluorescent acceptor conjugated to a glycosylated substrate. In another embodiment the sensor comprises a fluorescent donor conjugated to a glycosylated substrate. The invention further contemplates the mutant Concanavalin A comprises the polypeptide of SEQ ID NO:15.

### BRIEF DESCRIPTION OF THE DRAWINGS

FIG. 1 is a stereo diagram of dimeric ConA.

FIG. 2 shows the proteolytic maturation pathway for Concanavalin A.

FIG. 3 shows an SDS-PAGE gel of natural Concanavalin A (nConA) purified by NH₄HCO₃ precipitation.

FIG. 4 shows an SDS-PAGE gel of natural Concanavalin A (nConA) purified by NH₄HCO₃ precipitation and denaturing gel filtration chromatography.

FIG. 5 shows the nucleic acid sequence of mature Concanavalin A from C. ensiformis.

FIG. 6 shows the nucleic acid sequence of mature Concanavalin A from C. gladiata.

FIG. 7 shows the amino acid sequence of mature Concanavalin A from C. ensiformis.

FIG. 8 shows the amino acid sequence of mature Concanavalin A from C. gladiata.

FIG. 9 shows the amino acid sequence of pre-pro Concanavalin A.

FIG. 10 is a generalized diagram of splicing overlap extension PCR.

FIG. 11 shows the primer design for “mature” ConA SOE PCR

FIGS. 12A-12B show expression Conditions Optimized for rConA

FIG. 13 depicts and SDS-PAGE gel of rConA purified by Sephadex G-75 affinity chromatography.

FIGS. 14A-D shows FRET results for conjugated rConA.

FIG. 15 shows the binding of ConA to GPITC-tHSA.

FIG. 16 is a graphical representation of the HPLC data for purified recombinant Concanavalin A from C. gladiata (gConA).

FIG. 17 is a graphical representation of the HPLC data for purified recombinant mutant Concanavalin A (mConA).

FIG. 18 is an SDS-PAGE gel of gConA.

FIG. 19 is an SDS-PAGE gel of mConA.

FIG. 20 is a graphical depiction of the SEC-MALLS characterization of gConA.

FIG. 21 is a graphical depiction of the SEC-MALLS characterization of mConA

FIG. 22 is a graphical depiction of the SEC-MALLS characterization of mConA performed by a third-party.

FIG. 23 depicts FRET results for mConA in solution.

FIG. 24 depicts FRET results for gConA in solution.

FIG. 25 depicts FRET results for mConA in sensors.

FIG. 26 depicts FRET results for gConA in sensors.

FIG. 27 depicts comparative Biacore binding data for mConA and gConA.

FIG. 28 depicts an exemplar vector used to express Concanavalin A.

### DETAILED DESCRIPTION OF THE INVENTION

Definitions

Various terms relating to the biological molecules of the present invention are used throughout the specification and claims.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

“Polynucleotide” generally refers to any polyribonucleotide or polydeoxyribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA.

The term polynucleotide also includes DNA's or RNA's containing one or more modified bases and DNA's or RNA's with backbones modified for stability or for other reasons. “Modified” bases include, for example, LNA's, tritylated bases and unusual bases such as inosine. A variety of modifications can been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising at least two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the gene-encoded amino acids.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical

A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques or by direct synthesis. For instance, a conservative amino acid substitution may be made with respect to the amino acid sequence encoding the polypeptide.

Variant proteins encompassed by the present invention are biologically active, that is they continue to possess the desired biological activity of the native protein, as described herein. Such variants may result from, for example, genetic polymorphism or from human manipulation. Biologically active variants of a mConA protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

A “conservative amino acid substitution”, as used herein, is one in which one amino acid residue is replaced with another amino acid residue having a similar side chain. Families of amino acid residues having similar side chains have been defined in the art and are defined, for example, in M. J. Betts, R. B. Russell, Amino acid properties and consequences of substitutions, Bioinformatics for Geneticists, M. R. Barnes, I. C. Gray eds, Wiley (2003), which is hereby incorporated by reference.

The term “substantially the same” when referring to nucleic acid or amino acid sequences, refers to nucleic acid or amino acid sequences having sequence variations that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

With respect to single-stranded nucleic acid molecules, the term “specifically hybridizing” refers to the association between two single-stranded nucleic acid molecules of sufficient complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

With respect to oligonucleotide constructs, but not limited thereto, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide construct with a substantially complementary sequence contained within a single-stranded DNA or RNA molecule of the invention, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g., enhancers and regulators) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a vector for transforming a cell, in vitro or in vivo. This term may be used interchangeably with the term “transforming DNA”. Such a nucleic acid construct may contain a coding sequence for a gene product of interest, along with a selectable marker gene and/or a reporter gene.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The term “DNA construct”, as defined above, is also used to refer to a heterologous region, particularly one constructed for use in transformation of a cell. A cell has been “transformed” or “transfected” or “transduced” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

The term “recombinant” when made in reference to a DNA sequence refers to a DNA sequence which is comprised of segments of DNA joined together by means of molecular biological techniques. The term “recombinant” when made in reference to a polypeptide sequence refers to a polypeptide sequence which is expressed using a recombinant DNA sequence.

As used herein, the terms “vector” and “vehicle” are used interchangeably in reference to nucleic acid molecules that transfer DNA segment(s) from one cell to another.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

The term “recombinant ConA” or “rConA” is meant to refer to either the nucleic acid sequence or the polypeptide sequence of any mature form of Concanavalin A that has been derived from recombinant methods. In particular, it is meant to refer to recombinant Concanavalin A derived from C. ensiformis or C. gladiata, the polypeptide sequences of which are shown in SEQ ID NO:3 and SEQ ID NO:4 respectively.

The term “gConA” refers to recombinant Concanavalin A comprising the polypeptide sequence of SEQ ID NO:4 (C. gladiata).

The term “mConA” refers to a mutant Concanavalin A comprising the polypeptide sequence of SEQ ID NO:15.

Novel Polypeptides and Nucleic Acids

The invention contemplates both novel polypeptides and nucleic acids encoding said novel polypeptides.

I. Nucleic Acids

The nucleic acid molecules can be prepared by any suitable method. Two useful methods include synthesis from an appropriate nucleotide trios phosphates, and isolation from biological sources. These methods can utilize protocols that are well known in the art and examples of which are set forth herein.

The nucleic acid molecules include cDNA, genomic DNA, RNA, and fragments thereof, which may be single- or double-stranded. The oligonucleotides (sense or antisense strands of DNA or RNA, siRNA) have sequences that are capable of hybridizing with at least one sequence of a nucleic acid molecule of the present invention, such as selected segments of the cDNA having SEQ ID NO: 16.

The nucleic acids may be maintained as DNA in any convenient cloning vector. Clones can be maintained, for example, in a plasmid cloning/expression vector, examples of which are included below, the plasmid being propagated in a suitable host cell.

II. Polypeptides

Recombinant proteins and polypeptides of the present invention may be prepared in a variety of ways, according to known methods. For example a cDNA or gene encoding for the protein of the invention may be cloned into an appropriate transcription vector. A host cell may be transformed with the transcription vector and the protein expressed either intracellularly or extracellularly. In one aspect of the invention, the protein of the invention is expressed intracellularly, inclusion bodies are formed, the inclusion bodies and the protein of the invention are solubilized and the protein of interest is purified from solution.

A cell free system may also be used for protein production. A cDNA or gene, for example, may be cloned into an appropriate in vitro transcription vector, such as pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

Polypeptides can contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

III. Mutant Concanavalin A (mConA)

In particular, the invention includes both polypeptides and nucleic acids encoding polypeptides comprising one or more mutants of Concanavalin A having improved production and/or purification properties. More specifically, the invention includes mutations to the sequence encoding naturally occurring Concanavalin A that change one or more acidic amino acids to neutral amino acids. These mutations result in a protein with improved characteristics, including reduced precipitation of mConA during purification and conjugation to Cy dyes.

An exemplary mutant of C. ensiformis was produced wherein an acidic amino acid (aspartic acid) immediately adjacent to a lysine residue close to the glucose binding site (K59), was changed into a neutral amino acid (glycine). This mutation (D58G) converts this region of ConA from C. ensiformis (amino acids VDKRL, SEQ ID NO:19) into the sequence found in C. gladiata (amino acids VGKRL, SEQ ID NO:20).

This mutation improves rConA performance in both dye labeling and FRET reactions. In addition, this single amino acid mutation results in reduced precipitation of rConA during purification and conjugation to Cy dyes. The full length mutant ConA (“mConA”) polypeptide sequence is depicted in SEQ ID NO:15 and an exemplary nucleic acid sequence coding for the mConA polypeptide is depicted in SEQ ID NO:16.

The invention also contemplates mutants with other non-conservative amino acid substitutions to Concanavalin A near the glucose binding site (K59). In particular, the invention contemplates non-conservative amino acid substitutions at between the amino acid at positions 34 and the amino acid at position 64.

The invention also includes polypeptides having conservative amino acid substitutions to the aforementioned mutants as well as polypeptides having both conservative and non-conservative amino acid substitutions to the polypeptide of SEQ ID NO:15, as well as nucleic acid sequences encoding the aforementioned polypeptides.

Production and Purification of Lectins

The invention also includes an improved process for producing and purifying lectins, and in particular Concanavalin A (ConA), of relatively high purity. The invention also contemplates compositions comprising highly purified lectins. Historically, purifying ConA from natural sources has been difficult, resulting in a number of problems. These problems include, in the case of ConA, methods which produce compositions, which, after purification, contain full length and fragmented ConA monomers.

The invention contemplates a method of producing a recombinant lectin by inducing expression of the lectin in a bacterial cell culture that has been transformed by a vector containing a gene coding for the lectin of interest. The induction occurs in such a manner so as to encourage the formation of inclusion bodies. The cells of the bacterial culture are then lysed to produce an inclusion body fraction. The inclusion body fraction is then purified and the inclusion bodies are solubilized so that the lectin of interest is present in solution. The lectin is then denatured and subsequently allowed to re-fold in solution. The solution is then purified to recover the lectin of interest.

By way of example, the process of the invention includes using vectors having an antibiotic resistance gene coupled to a promoter and a gene for recombinant Concanavalin A (rConA). In another example, the process of the invention includes using vectors having an antibiotic resistance gene coupled to a promoter and gene for mConA. Antibiotic resistance genes include, for example, ampicillin, kanamycin, and tetracycline.

The transformed bacterial cells can be induced either in the presence or absence of antibiotic. For example, the transformed bacterial cell culture can be induced with isopropyl β-D-thiogalactopyranoside (IPTG) in the absence of kanamycin.

Solution purification can be performed by a number of different methods, including but are not limited to affinity chromatography and size-exclusion chromatography. In one example, affinity chromatography alone is used to purify the solution. In another example, both affinity chromatography and size-exclusion chromatography are used.

The production process is useful for producing a recombinant Concanavalin A including, e.g., gConA and mConA. This production and purification process results in highly purified protein, particularly highly purified recombinant protein including, e.g., lectins having a purity of at least about 95%, at least about 96%, at least about 97%, at least about 98%, and at least about 99%.

The 26 kDa Concanavalin A monomer is preferably substantially free of contaminants including e.g., contaminants having molecular weights from about 10 kDa to about 20 kDa, from about 30 kDa to 40 kDa (as determined under reducing, denaturing conditions via SDS-PAGE as disclosed herein), or combinations thereof. Our purification method has produced ConA of sufficient purity, e.g., ConA having a level of contaminants of less than 5%, less than about 4%, less than about 3%, less than about 2%, and less than about 1%.

Sensors

The invention also includes sensors having a purified recombinant lectin. The sensors are capable of detecting the presence of an analyte. The sensors include a reagent suitable for detecting the analyte in a liquid, e.g., body fluid (e.g., blood and interstitial fluid). Useful reagents include, e.g., energy absorbing reagents (e.g., light absorbing and sound absorbing reagents), x-Ray reagents, spin resonance reagents, nuclear magnetic resonance reagents, and combinations thereof.

A useful class of reagents includes fluorescence reagents, i.e., reagents that include a fluorophore or a compound labeled with a fluorophore. The fluorescence reagent can reversibly bind to the analyte and the fluorescence behavior of the reagent changes when analyte binding occurs.

Changes in fluorescence associated with the presence of the analyte may be measured in several ways. These changes include changes in the excited state lifetime of, or fluorescence intensity emitted by, the fluorophore (or component labeled with the fluorophore). Such changes also include changes in the excitation or emission spectrum of the fluorophore (or component labeled with the fluorophore). Changes in the excitation or emission spectrum, in turn, may be measured by measuring (a) the appearance or disappearance of emission peaks, (b) the ratio of the signal observed at two or more emission wavelengths, (c) the appearance or disappearance of excitation peaks, (d) the ratio of the signal observed at two or more excitation wavelengths or (e) changes in fluorescence polarization.

The reagent can be selected to exhibit non-radiative fluorescence resonance energy transfer (FRET), which can be used to determine the occurrence and extent of binding between members of a specific binding pair.

Examples of FRET, FRET-based sensors, their use and method of manufacture, are described in U.S. Pat. No. 6,040,194 and U.S. Publ. No. 2005-0095174, filed Oct. 31, 2003 which are hereby incorporated by reference in their entirety. Examples of other sensors are also described in U.S. Pat. Nos. 6,319,540, 6,383,767, 6,850,786, and 5,342,789, which are also hereby incorporated by reference.

The sensors of the invention can be implantable. The implantable sensor may be provided with a selectively permeable membrane that permits the analyte (but not fluorescence reagent) to diffuse into and out of the sensor. In another embodiment, at least some of the components of the fluorescence reagent are immobilized within the sensor (e.g., on a substrate or within the pores of a porous matrix). For example, in the case of an analogue labeled with donor and a ligand labeled with acceptor, one (or both) materials can be immobilized. In another embodiment, at least some of the components of the fluorescence reagent are freely mobile (i.e., not immobilized) within the sensor.

The sensors of the invention include: sensors made with conjugated pairs of rConA and Human Serum Albumin (“HSA”); and sensors made with mConA and HSA are contemplated.

The sensors of the present invention can be used to detect a wide range of physiological analyte concentrations (e.g., concentrations ranging from 0.5 to 18 mg/ml in the case of glucose).

One skilled in the art will appreciate further features and advantages of the invention based on the above-described embodiments. Accordingly, the invention is not to be limited by what has been particularly shown and described, nor by the examples set forth below, except as indicated by the appended claims. All publications and references cited herein are expressly incorporated herein by reference in their entirety.

### EXAMPLES

I. Expression and Purification of Recombinant ConA (rConA)

A. Cloning Mature ConA Coding Region

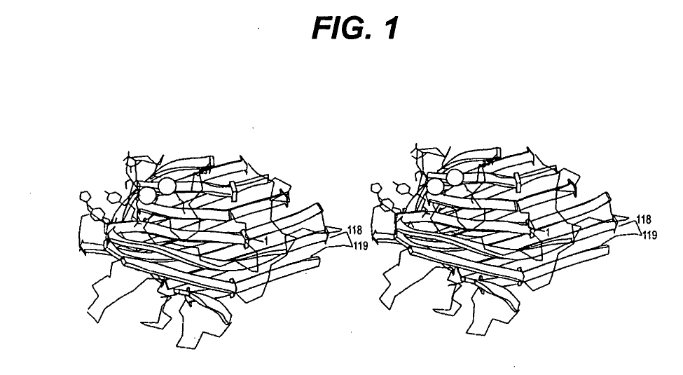

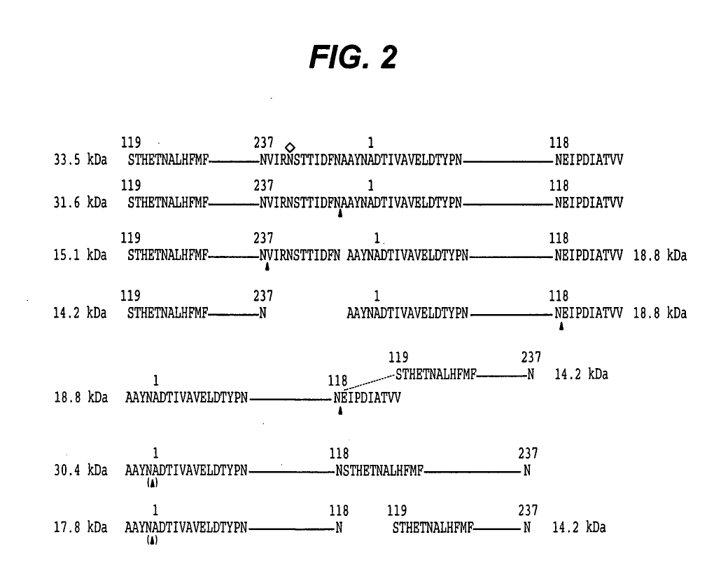

Due to the post-translational modifications necessary for producing “mature” ConA, cloning the DNA coding region for “mature” ConA is challenging. ConA maturation requires a series of proteolytic digestions followed by transpeptidation of the N-terminal and C-terminal halves of a non-functional precursor (pre-pro-ConA) (FIG. 1b, Carrington, et. al., 1985). From a cloning perspective, the result is a primary amino acid sequence that does not correspond to the predicted amino acid sequence derived from the genomic ConA coding region. This prevents direct cloning of the “mature” ConA coding region from natural DNA sources.

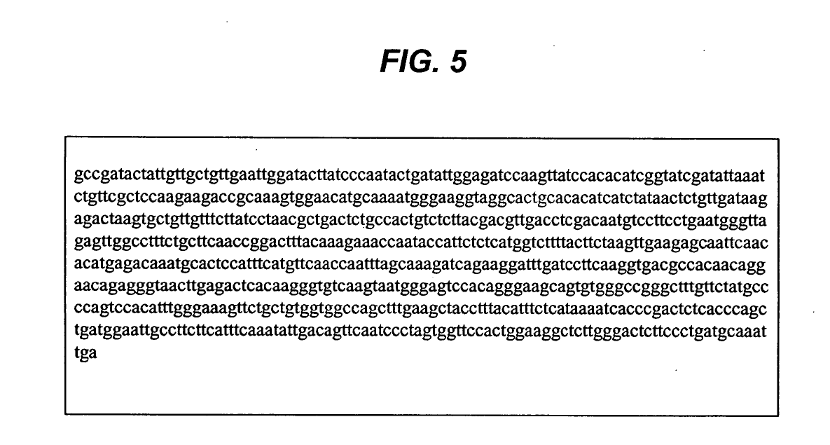

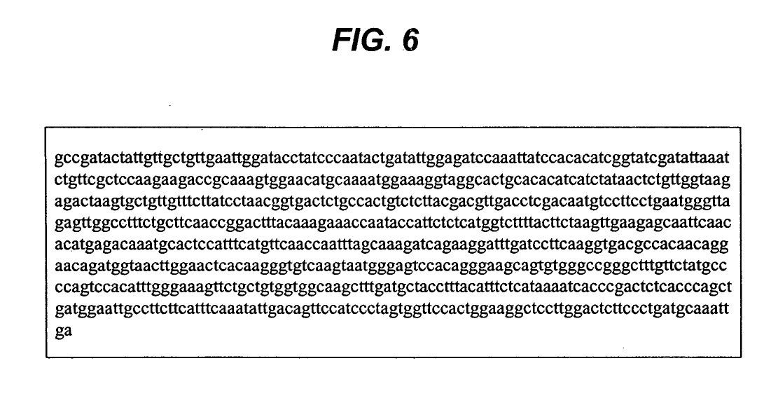

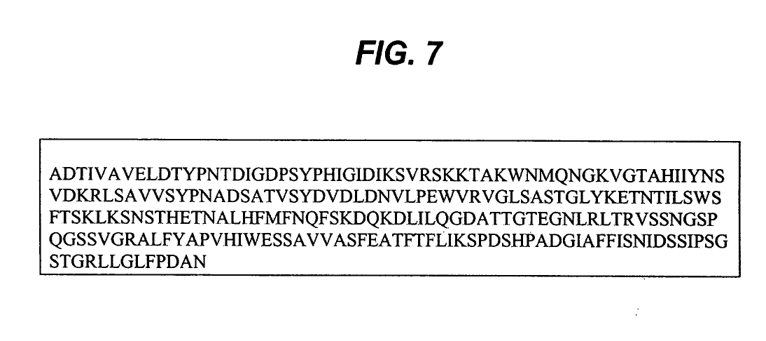

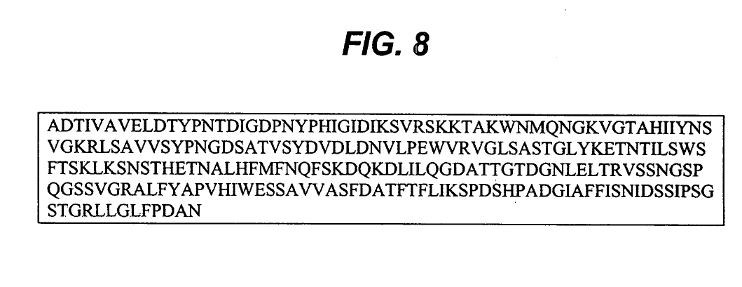

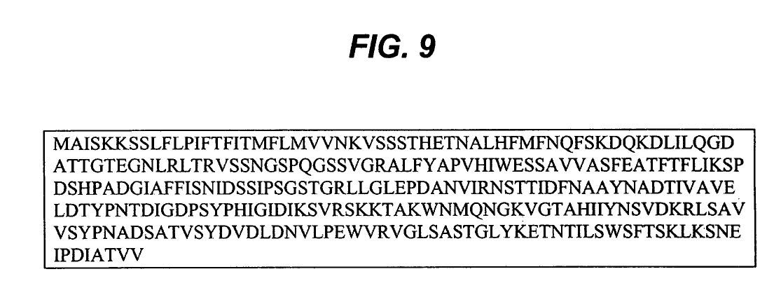

In lieu of directly cloning the “mature” ConA coding region suitable for expression, the “mature” ConA DNA sequence was assembled based on genomic, precursor DNA sequences. Construction of the mature ConA coding region required isolating and rearranging those sections of preConA DNA which code for the mature ConA primary amino acid sequence (FIGS. 7 and 8). FIGS. 5 and 6 show the mature DNA sequence deduced from the preConA DNA sequences from two plant species (C. ensiformis sequence derived from Carrington, et al. Nature 313:64 1985; and C. gladiata sequence derived from Yamauchi, et al. FEBS Lett. 260:127 1990). FIGS. 7 and 8 show the corresponding amino acid sequence of mature C. ensiformis (AA seq from Carrington, et al.) and amino acid sequence of mature C. gladiata (AA seq from Yamauchi, et al). The two deduced “mature” ConA DNA sequences (FIGS. 5 and 6) were used to design and construct recombinant ConA expression systems.

B. Construction of ConA cDNA

i. Gene Synthesis Technology

Due to the extensive DNA rearrangement required to design a mature ConA coding region, there were a limited number of methods to construct a “mature” ConA cDNA for bacterial expression. One method utilized gene synthesis technology in which single nucleotides were ligated chemically according to a predesigned DNA sequence. This procedure was analogous to methods used in the synthesis of oligonucleotides. There were several benefits in using gene synthesis as a means for cDNA construction. First, the ease in which coding regions for chimeric proteins (e.g. mature ConA) could be synthesized. Second, coding regions could be optimized for codon usage in any host expression system to maximize recombinant protein expression. Finally, restriction sites could be engineered anywhere for future cloning purposes.

Gene synthesis of mature ConA was performed by the company GeneArt (Germany). Initially, the “supersized” ConA oligonucleotide synthesized was based on the sequenced outlined herein (FIGS. 5 and 6). In addition, several modifications were made to the ConA coding region. First, the synthesized ConA gene was optimized for codon usage in E. coli. Second, NcoI and BamH1 restriction sites were engineered at 5′ and 3′ ends, respectively, for cloning into the bacterial expression vector pET15b. Finally, the secretion signal sequence from the E. coli outer membrane protein (ompA) was engineered at the 5′ end of the ConA coding sequence.

ii. cDNA Cloning from Jack Bean

Another method for constructing a mature ConA cDNA, was through direct cloning of the precursor ConA coding region from Canavalia ensiformis beans (jack beans). This was a multistep cloning process requiring the synthesis of pre-ConA cDNA from isolated jack bean RNA, cloning of preConA cDNA, and genetic rearrangement of the pre-ConA coding region by PCR to generate the mature ConA coding region. The result would be a single cDNA clone that codes for mature ConA identical to that obtained with gene synthesis.

(a) Synthesis and Purification of Jack Bean Total RNA and cDNA

Mature ConA cDNA was synthesized from total jack bean cDNA derived from purified RNA. Immature jack beans (0.5 kg) were harvested from ˜6 week old Canavalia ensiformis plants (Plantwise Enterprises). The beans were rapidly frozen in liquid nitrogen, to preserve the beans and eliminate all RNase activity, and stored at −80° C. A single jack bean (˜1.8 g) was crushed using a pre-chilled mortar and pestle pre-treated with RNAZap from Ambion. A 1 ml pipette tip was used to scoop ˜⅓ of the crushed meal and transferred to a sterile microfuge tube (˜250 ul in volume). Total RNA was isolated from the meal using RNAqueous total RNA isolation kit and Plant Isolation Aid (Ambion) according to manufacturers conditions. Approximately 50 ug of total jack bean RNA was isolated by this method.

(b) Isolation and Cloning of Precursor ConA cDNA (Pre-Pro ConA)

Next, pre-ConA cDNA was purified and cloned into a conventional sequencing vector. An aliquot of purified total RNA (4 ug) was first reverse transcribed with MMLV reverse transcriptase using Retroscript (Ambion) to generate an aliquot of total jack bean cDNA. Gene specific isolation of the pre-pro ConA cDNA was then achieved by polymerase chain reaction (PCR). Three gene specific forward primers and one reverse primer, based on the pre-pro ConA sequence (Genbank), were designed using the Primer Premier software suite (Biosoft International) (Table. 1). Three PCR reactions using each primer pair and 5 ul of total cDNA were set up using an Eppendorf Mastercycler and employing the Touchdown PCR conditions outlined in Table. 2. PCR reaction efficiencies were assessed by agarose gel electrophoresis. PCR products corresponding to the correct approximate molecular weight of pre-pro ConA cDNA (˜950 bp) were band purified by preparative gel electrophoresis and isolated using Zymoclean (Zymo Research). Gel purified PCR products were cloned into the sequencing vector pCR2.1 using TOPO TA cloning kit for sequencing (Invitrogen).

TABLE 1
Pre-pro ConA PCR primers

| | | |
|---|---|---|
| Direction | Name | Sequence |

| | | |
|---|---|---|
| Sense | 5′preConA1 | 5′ATTGTAGCAAGCAGCACTAC3′ |
| Sense | 5′preConA2 | 5′TAGCAAGCAGCACTACTAGTG3′ |
| Sense | 5′preConA3 | 5′GCAAGCAGCACTACTAGTGA3′ |
| Anti- | 3′preConA | 5′GAGATTATTATGGTACATGGATGA3′ |
| sense | | |

TABLE 2
Pre-pro ConA PCR conditions

| Stage | Temperature | Time (minutes) | # of cycles |
|---|---|---|---|
| Initial denature | 94° C. | 2.0 | 1 |
| Denature | 94° C. | 0.5 | 5 |
| Annealing | 51° C. | 0.5 | 5 |
| Extension | 72° C. | 1.0 | 5 |
| Denature | 94° C. | 0.5 | 5 |
| Annealing | 48° C. | 0.5 | 5 |
| Extension | 72° C. | 1.0 | 5 |
| Denature | 94° C. | 0.5 | 25 |
| Annealing | 45° C. | 0.5 | 25 |
| Extension | 72° C. | 1.0 | 25 |
| Extension | 72° C. | 5.0 | 1 |
| Hold | 4° C. | overnight | 1 |

(c) Verification of Pre-Pro ConA cDNA Sequence

Next, DNA sequencing analysis was employed to ensure the cloned PCR products corresponded to the published pre-pro ConA sequence. Two independent clones containing the PCR product were isolated and analyzed by DNA PCR cycle sequencing (University of Massachusetts Medical School Nucleic Acid Facility). Two sequencing primers (M13 universal and M13 reverse) were used in separate sequencing reactions to sequence the entire cloned DNA. DNA sequence data from the reactions were received as ABI (Applied Biosystems) chromatograms and analyzed using the Sequencher software suite (Gene Codes Corporation). The DNA sequence of the cloned PCR products completely matched the Genbank published sequence using the BLAST DNA alignment algorithm (NCBI).

iii. “Mature” ConA cDNA Synthesis by SOE PCR

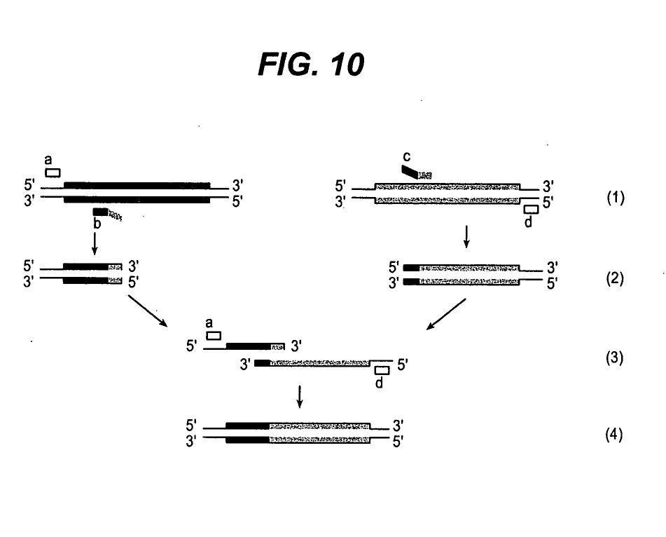

With the pre-pro ConA cDNA cloned, the mature ConA coding region was generated using a specific PCR method known as gene splicing overlap extension (SOE PCR). SOE PCR is a PCR procedure used for the creation of novel genes including chimeric proteins (Warrens, et. al., 1997; Lefebvre, et. al., 1995). With SOE PCR, PCR primers were specifically designed to unique regions of a target sequence to add, delete, or rearrange any portion of the DNA (FIG. 10). This type of genetic rearrangement required two sequential PCR reactions and four PCR primers, two of which were almost completely complementary (FIG. 10). The first series of PCR reactions produced DNA products that were complementary within a specific region that creates the chimera. To endfill the uncomplemented regions, the annealed PCR products were used as the template for the second PCR reaction to complete the final chimeric product.

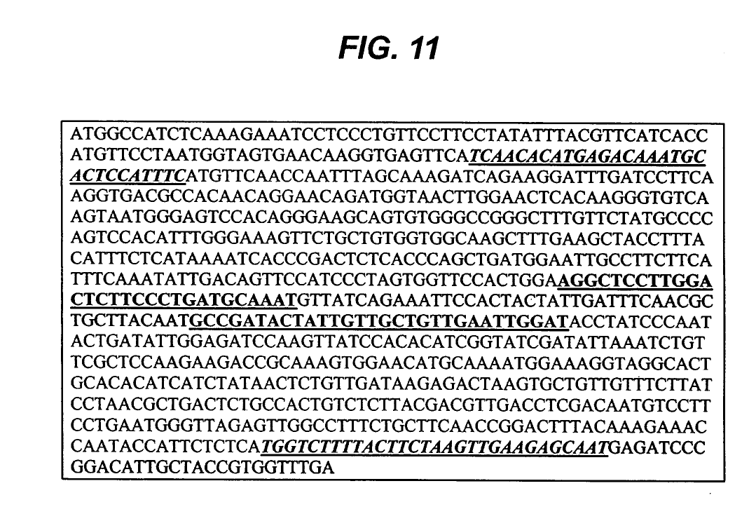

To use SOE PCR for synthesizing mature ConA cDNA, the mature DNA sequence (FIGS. 5 and 6) was compared to the pre-pro ConA sequence to devise a PCR primer strategy. As stated above, one of the primary modifications of preConA maturation is a transpeptidation reaction which entails the switching and re-ligation of the C-terminal and N-terminal halves of the protein. The initial strategy was to deduce those regions of pre-pro ConA involved in the transpeptidation reaction at the DNA level. FIG. 11 illustrates those portions of the pre-pro ConA cDNA which constitute “mature” ConA. The coding region from B1 to B2 is the N-terminal half while the sequence from A1 to A2 represents the C-terminal half. Four PCR primers were design using Primer Premier that are complementary to those regions involved in the transpeptidation reaction (Table. 3). One primer pair was directed towards the N-terminal half of mature ConA (ConApt1 (C and D)) while the second primer pair generated the C-terminal half (ConApt2 (A and B)). The overlapping primers (ConApt1(D) and ConApt2(A), FIG. 11, Table 3) facilitated the synthesis of the final mature ConA product by mimicking the transpeptidation reaction at the DNA level.

TABLE 3
Primer pair sequences for ConA SOE PCR (1^{st} round)

| | | |
|---|---|---|
| Direction | Name | Sequence |

| | | |
|---|---|---|
| Sense | ConApt1(C) | 5′GCCGATACTATTGTTGCTGTTGAATTG |
| | | GAT3′ |
| Anti- | ConApt1(D) | 5′GAAATGGAGTGCATTTGTCTCATGTGT |
| Sense | | TGAATTGCTCTTCAACTTAGAAGTAAAAG |
| | | ACCA3′ |
| Sense | ConApt2(A) | 5′TGGTCTTTTACTTCTAAGTTGAAGAGC |
| | | AATTCAACACATGAGACAAATGCACTCCA |
| | | TTTC3′ |
| Anti- | ConApt2(B) | 5′TCAATTTGCATCAGGGAAGAGTCCAAG |
| Sense | | GAGCCT3′ |

The conditions used for SOE PCR of the mature ConA cDNA are outlined below. Purified pCR2.1-preConA was used as the template in the first series of PCR reactions. The PCR products (˜350 bp each) from each reaction were purified by agarose gel electrophoresis and extracted using Zymoclean (Zymo Research). The second PCR reaction (primers ConApt1(C) and ConApt2(B) plus the annealed PCR product as template) resulted in a ˜700 base pair product, approximately the predicted size for mature ConA cDNA. The PCR product was purified by agarose gel electrophoresis, extracted using Zymoclean (Zymo Research) and cloned into the pCR2.1 sequencing vector (TOPO TA cloning kit for sequencing, Invitrogen). Confirmation of successful mature ConA cDNA synthesis was determined by DNA PCR cycle sequencing (University of Massachusetts Medical School Nucleic Acid Facility). Two sequencing primers (M13 universal and M13 reverse) were used in separate sequencing reactions to sequence the entire cloned DNA. DNA sequence data from the reactions were received as ABI (Applied Biosystems) chromatograms and analyzed using Sequencher software suite (Gene Codes Corporation). The DNA sequence of the cloned PCR products completely match the mature ConA sequence defined in herein using the BLAST2 DNA alignment algorithm.

C. Expression of Recombinant ConA

i. Selection of Bacterial Expression System

Any suitable expression system can be used. Useful expression systems include e.g., cell free translation systems, as well as, cell based translation systems (e.g., mammalian, yeast, insect, bacterial). Bacterial expression systems provide for both soluble and insoluble expression. A specific example of a suitable expression system includes an E. coli based system, which directs the expressed proteins into inclusion bodies. Inclusion bodies can be utilized for the enrichment of expressed recombinant protein. By using specific growth conditions and expression system components that force synthesized recombinant proteins into inclusion bodies, the recombinant protein of interest was easily harvested by simple, centrifugal fractionation procedures.

To ensure the production of inclusion bodies composed solely of insoluble rConA, the secretion signal sequence of the E. coli outer membrane protein (ompA) was used to facilitate ConA enrichment. The ompA DNA signal sequence was ligated to the 5′ end of the mature ConA sequence by both gene synthesis and DNA recombinant technology to facilitate rConA purification.

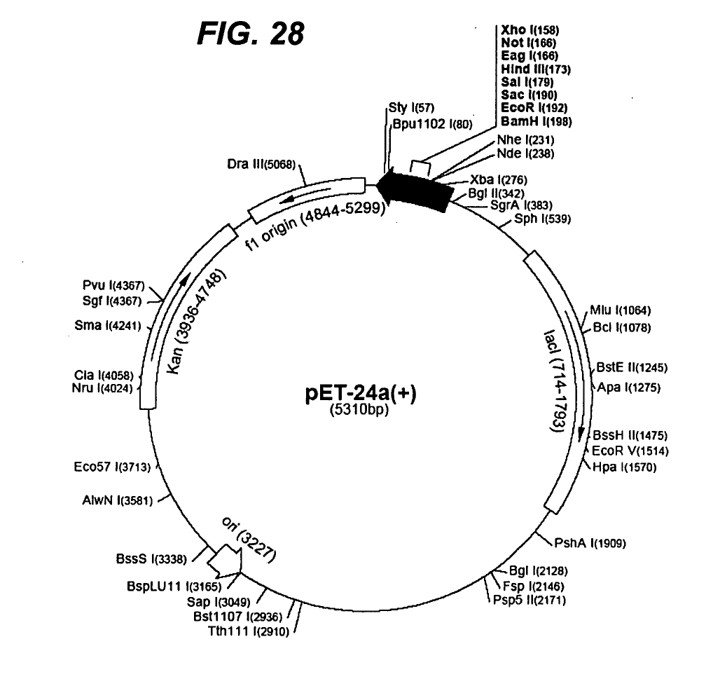

The pET15b vector, which contains an ampicillin resistance gene was predominantly used for the cloning and expression of naturally occurring recombinant ConA (gConA). A mutant form of ConA (mConA), more fully described below, was also cloned and expressed. The pET24b plasmid (FIG. 28), which carries a kanamycin resistance gene was used to express mConA.

ii. Bacterial Expression Conditions

(a) Selection of E. coli Strain

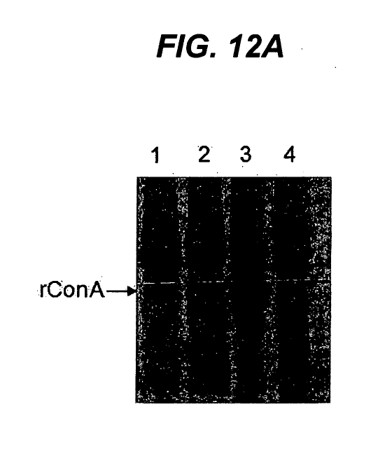

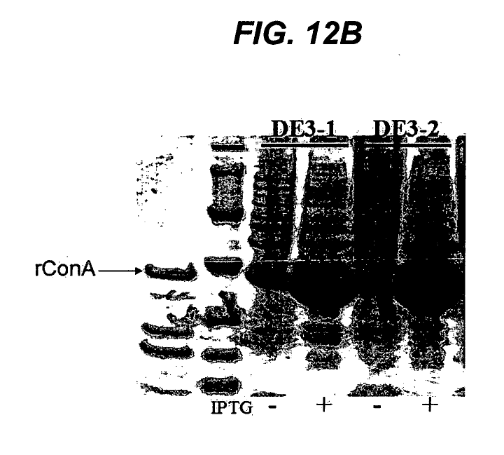

Two common E. coli strains for T7 RNA polymerase-based expression systems, BL21(DE3) and BL21(DE3)pLys were used. Expression of rConA using BL21(DE3) and BL21(DE3)pLys strains of E. coli were compared to optimize for levels of expression. Small-scale bacterial expression (<50 ml) was used to express rConA in BL-21 (negative control), BL21(DE3) and BL21(DE3)pLys. Isolated inclusion bodies were resuspended in SDS sample buffer and boiled at 95° C. for 5 minutes and analyzed on SDS-PAGE. Ten μl of sample extract was loaded in each gel well. FIG. 12a shows the relative amount of rConA expressed and localized to inclusion bodies for BL21(DE3) (Lane 3), BL21(DE3)pLys (Lanes 1 and 2) and BL21 (Lane 4, negative control). Since expression levels of rConA were highest in BL21(DE3), this bacterial strain was selected for subsequent expression of rConA.

(b) Specific Induction of rConA Expression in DE3

Two BL21(DE3) clones expressing rConA were selected to characterize specific induction by isopropyl β-D-thiogalactopyranoside (IPTG). Small-scale bacterial expression (<50 ml) was used to express rConA in two BL21(DE3) clones, DE3-1 and DE3-2. Isolated inclusion bodies were resuspended in SDS sample buffer and boiled at 95° C. for 5 minutes and analyzed on SDS-PAGE. FIG. 12b shows specific induction of rConA in the presence (+) of IPTG for both DE3-1 and DE3-2 clones expressing rConA. Since both DE3-1 and DE3-2 exhibited IPTG dependent induction of rConA expression, both clones were used for subsequent expression of rConA from C. ensiformis.

FIG. 12 shows expression conditions optimized for rConA. (A) SDS-PAGE of expression rConA in BL21(DE3)pLys (Lanes 1-2), BL21(DE3) (Lane 3) and BL21 (Lane 4, negative control). Ten (10) ul extracts from inclusion bodies of each induction culture was run on 10% Bis-Tris acrylamide gel and stained with colloidal blue stain (Simply Blue, Invitrogen). (B) SDS-PAGE of BL21(DE3) rConA clones (DE3-1 and DE3-2) in the presence (+) and absence (−) of IPTG. 10 ul extracts from inclusion bodies of each induction culture was run on 10% Bis-Tris acrylamide gel and stained with colloidal blue stain (Simply Blue, Invitrogen).

(c) Effect of Temperature on rConA Expression

Localization of rConA in soluble and insoluble (inclusion bodies) fractions during expression in E. coli is dependent on temperature (Min, et. al., 1992). To select the optimal temperature for rConA expression, small-scale bacterial cultures (<50 ml) were induced at two temperatures, 30° C. and 37° C. Subsequent purification efforts utilized 37° C. for bacterial growth and induction, and focused on proper refolding and affinity purification of expressed rConA.

C. Production and Purification of Recombinant ConA

i. Construction of Induction Cultures

Two induction cultures were grown over a 48-hour period. The first culture consisted of the inoculation of single 5 ml 2XYT culture with either a single bacterial colony (BL21(DE3)) containing pETConA plasmid) or directly from frozen glycerol stock. The following day, 25 ul of the overnight culture was used to inoculate a 25 ml 2xYT culture, which was shaken overnight at 37° C. in an incubator.

ii. Induction

To induce expression of rConA, 5 ml of overnight culture was used to inoculate 1 L of 2xYT culture (1 L per 2 L flask-4 L total). The culture then grows for 2.5 hours at 37° C. in a shaking incubator. For maximal protein expression, bacterial cultures were induced during the logarithmic phase of the growth cycle. The optical density of the culture at 600 nm was determined with a spectrophotometer. Typically, optical density of a logarithmically growing culture is between 0.5 and 0.8. Once the culture has reached the appropriate optical density, 119 mg of isopropyl β-D-thiogalactopyranoside per liter of log phase culture was added to a final concentration of 0.5 mM. The induced culture incubates at 37° C. in shaking incubator for additional 3 hours. At the end of the induction period, the culture was centrifuged and the bacterial pellets stored overnight at −80° C.

iii. Inclusion Body Purification

The frozen bacterial pellets were resuspended in 400 ml of ConA lysis buffer (20 mM MOPS, 1M NaCl, 5 mM EDTA, 0.5% Triton X-100, 0.01% sodium azide, 1 mg/ml lysozyme, 10 mM (10 mM), 5 ug/ul DNase I pH 7.0) to release the inclusion bodies. 25 ml of the resuspended pellet was aliquoted into eight 35 ml Oak Ridge tubes. The suspension was incubated rotating for 20 min at room temperature. To shear residual chromosomal DNA and lyse any remaining intact cells, lysates were sonicated for 30 seconds to 1 min on ice. The insoluble protein fraction was subsequently isolated by centrifuging the lysates at high speeds (10,000 rpm) at 4° C.

To further purify the inclusion body fraction, the insoluble pellet underwent several washing steps to remove any contaminating soluble proteins and other cellular debris. Inclusion body pellets were resuspended in 100 ml of ConA lysis buffer (without lysozyme/DNaseI) via brief sonication (30 sec). The resuspended pellets were centrifuged at 12,000 rpm at 4° C. This process was repeated 2× more with ConA lysis buffer (3× total). To remove detergent from the inclusion body pellet, the pellet was washed with 100 ml of Con A wash buffer (ConA lysis buffer without Triton X-100). Finally, to prepare for the denaturation/renaturation step of the purification procedure, EDTA was removed to allow the refolded rConA to coordinate Mn2+ and Ca2+ for proper function. To achieve this, the inclusion body pellet was washed a final time in ConA metals buffer (20 mM MOPS, 1M NaCl, 1 mM manganese chloride, 1 mM Calcium chloride, pH 7.0. The purified inclusion body pellets were frozen in liquid nitrogen and stored at −80° C.

iv. Denaturation/Renaturation Recombinant ConA

Purified inclusion bodies were thoroughly solubilized and rConA was allowed to refold slowly. Inclusion body pellets were solubilized and rConA denatured by adding 20 ml ConA denaturing buffer (containing 6M guanidine hydrochloride) per liter of culture followed by brief (10-20 sec.) sonication. The partially solubilized pellets were incubated at room temperature for two hours with slow rotation. At this point, the suspension was centrifuged at 12,000 rpm for 20 minutes at 4° C. to remove any insoluble material. The supernatant was passed 2× through a 1 ml DEAE-Sephacel column pre-equilibrated with ConA denaturing buffer to remove any insoluble materials. Upon loading the DEAE-Sephacel column a second time, the flowthrough was diluted 30-fold to initiate refolding of rConA.

The flow rate from the column was about 1 ml-2 ml per minute to allow thorough mixing of denatured rConA in the dilution buffer. The diluted protein solution was then stirred at 4° C. overnight to ensure proper refolding and the formation of intact tetramers.

v. Affinity Purification

The clarified protein solution was loaded onto a 40 ml Sephadex G75 column pre-equilibrated with ConA metals buffer at a flow rate of approximately 2.25 ml/min. (An alternative method was to batch purify the rConA tetramers by incubating the protein solution overnight at 4° C. with 10 ml Sephadex G-75). The column was immediately washed 2× with 400 ml ConA metals buffer. Bound protein was eluted by resuspending the matrix in 100 ml ConA elution buffer containing 20 mM methyl α-D-mannopyranoside. The protein concentration of each eluate was calculated (see below). Once determined, the eluates were pooled and stored at 4° C.

II. Purification of Natural ConA (nConA)

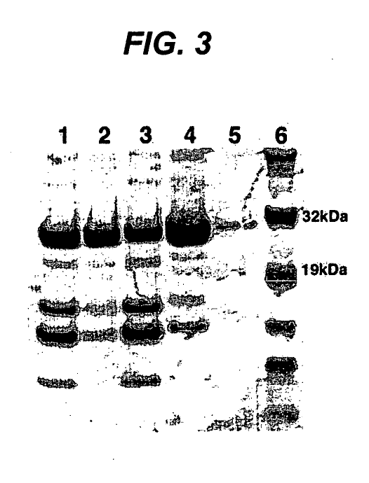

Natural ConA was purified using a modification of the method of Cunningham, et. al. A 10 mg/ml solution of natural ConA was re-suspended in 1% ammonium bicarbonate, pH 8.0 at 37° C. for 18 hours. The suspension was centrifuged at 12 k rpm and supernatant loaded on 1 ml Sephadex G-75 column. Twenty (20) ul from each stage was run on 10% Bis-Tris acrylamide gel and stained with colloidal blue (Simply Blue, Invitrogen). FIG. 3 shows the results using gel electrophoresis; Lane 1, unpurified nConA. Lane 2, NH4HCO3 supernatant. Lane 3 NH4HCO3 pellet. Lane 4 Sephadex G-75 eluate #1. Lane 5 Sephadex G-75 eluate #2. Lane 6 MW markers. This method resulted in enrichment for homotetrameric ConA in the purified supernatant. This differential precipitation technique resulted in ˜93.5% pure homotetrameric ConA when combined with a Sephadex G-75 affinity chromatography step to ensure purification of active ConA tetramer.

Purification of full-length, natural ConA monomers was also accomplished through the complete denaturation and reassembly of natural ConA homotetramers using size exclusion chromatography. Extremely harsh biochemical conditions are necessary for the disassembly and denaturation of ConA tetramers (Auer, et. al., 1971; Auer, et. al., 1984; Huet, et. al., 1975). ConA tetramers assemble in a pH dependent manner, forming stable tetramers between pH 7.0-7.5. Multimeric complexes consisting of high molecular weight aggregates occur at pHs greater than 7.5. Between pH 5-7 ConA disassembles into dimers and only below pH 5, ConA in solution is primarily monomeric. To ensure complete dimer dissociation, a two-component buffer was utilized: a low pH buffer (glycine based pH 3) to generate ConA monomers, and a chaotropic agent (8M Urea) to denature the monomers. Supernatant from NH4HCO3 precipitation was dialyzed against 8M Urea denaturing buffer, and the eluent concentrated to a final volume of 5 ml. The linear ConA polypeptide chains were purified to near homogeneity by size exclusion chromatography (Abe, et. al., 1971).

Two (2) ml of concentrate was loaded on a Sephacryl S-100 column pre-equilibrated with 8M Urea denaturing buffer. Fractions corresponding to ConA 26 kDa polypeptide were collected and pooled (50-fractions, 1 ml/each, flow rate of 0.5 ml/min). The pooled fractions revealed a 1.7 fold enrichment representing 90% of the total protein as shown by SDS-PAGE analysis (FIG. 3, lane 2). The remaining protein represents the 12 kDa fragment.

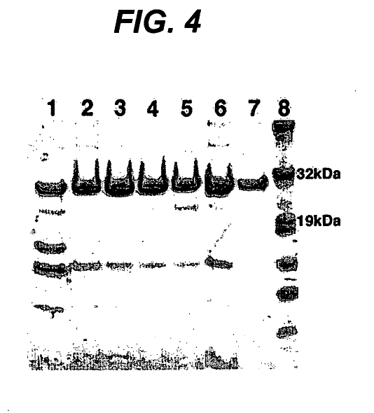

To reassemble ConA tetramers, denatured samples were diluted 30 fold in renaturation buffer (pH7.0 with Mn2+ and Ca2+) and purified by affinity chromatography (Sephadex G-75). Tetramers purified by this protocol were composed solely of 26 kDa monomer with no detectable levels of contaminating protein bands as demonstrated by gel electrophoresis (FIG. 4, Lane 1, unpurified nConA. Lanes 2-5, Sephacryl S-100 peak fractions. Lane 6, pooled gel filtration peak. Lane 7 Sephadex G-75 eluate. Lane 8, MW markers).

This method was not only applicable to the purification of natural ConA but may be used, generally, to purify lectins from various sources, including Concanvalin A from recombinant sources.

III. Protein Characterization

A. Concentration Determination and Purity Analysis

i. UV Analysis

Two analytical assays were conducted to determine the protein concentration, percentage yield, and purity of the purified material, for the ConA monomers. To monitor the purification process, aliquots were removed at all stages of purification starting at the inclusion body purification steps. To determine the protein concentration of the rConA eluates the absorbance of undiluted eluate at wavelength 280 nm was determined using a spectrophotometer. The values generated were used to calculate the concentration using the extinction coefficient for ConA. (OD280 ˜1.14=1 mg/ml ConA).

Percentage yield was calculated to determine amount of recoverable rConA during the purification procedure. To calculate this value, the concentration of the eluate was divided by the concentration of the starting material. After the refolding and clarification steps, the absorbance of undiluted, refolded rConA at 280 nm was determined and the concentration of the starting material calculated as described above. The percentage yield was computed by calculating the ratio of the eluate and total rConA concentrations

ii. SDS PAGE

As a final analytical step, the purity of rConA purified was determined both qualitatively and quantitatively. Qualitative analysis entailed visualizing the amount of 26 kD rConA monomer present by SDS-PAGE.

All rConA solutions were diluted in equal volumes of Sample Buffer (2×). The sample solutions were then heated for 10±1 minutes at 95±5° C. After cooling to room temperature, the samples were loaded on the gel. Analytical tests were conducted using NuPAGE® 10% Bis-Tris gels in the Xcell SureLock® Mini-Cell. The gels were loaded with 20 μL of the samples (5 μL of the marker). The gel rinsing, staining, and destaining steps all required 100 mL of the respective solutions. The gels were scanned and quantitated using the Bio-Rad Model Gel Doc® EQ Imaging System.

(a) Reaction Conditions

The final concentration of the reducing agent in the sample solution was IX. The marker used was Sigma Wide Range Molecular Weight Marker (M4038), that consists of thirteen protein bands. The NuPAGE® running buffer with 2-Morpholinoethanesulfonic acid (MES) was used. The gel was run at a voltage of 150 V. The run time was ˜1 hour. In the SDS removal step the gel was rinsed in water two more times, for 5 minutes. The protein bands on the gel were stained with SimplyBlue® SafeStain. The gel was then stained for a period of 1 hour. De-staining of the gels in water was then performed to reduce background and bring out the intensity of the bands of interest significantly. This process required a minimum of 2 hours but could be left overnight without compromising the results.

(b) Collection of Data for Establishment of Method Performance Criteria:

Performance criteria for use as the basis for establishing acceptance criteria were collected. In particular, Linearity, Precision, Specificity, and Accuracy data were collected.

Linearity was determined as follows: A gel was loaded with 8, 9, 10, 11, and 12 μg of rConA. The Trace Quantity for each of the major bands was plotted against the amount of rConA loaded on the gel. The correlation coefficient (r) of the linear least squares fit was ≧0.95.

Precision was determined as follows: A 10 μg load of rConA was used to determine the area percent of the major band (˜26 kDa) over a four-month period (each time with a different gel). The % RSD for the area percent of the major band of rConA was <1.0%.

System Suitability was determined as follows: The gel was loaded with 5 μL of M4038. Observation of thirteen well-resolved protein bands in M4038 indicated the system was suitable.

Specificity was determined by loading the following onto a gel and performing electrophoresis: Concanavalin A, Recombinant (10 μg of rConA); Bovine serum albumin (10 μg of P0914 054K8801); Spiked protein sample (10 μg of rConA+5 μg of P0914)

The rConA band had good separation from the major P0914 band (˜62 kDa) in the spiked protein sample. The rConA band of the spiked protein sample also lined up with the band of the rConA (˜26 kDa) of the pure sample thereby indicating specificity. The rConA band of the spiked protein sample also showed distinct separation from the P0914 band.

Accuracy was determined using a calibration curve obtained by plotting the Trace Quantity of each major band obtained against the respective amounts of rConA loaded (8, 9, 10, 11, 12, 14, 18, and 20 μg). Lanes on the same gel were also loaded with 10 μg of rConA spiked with 2, 4, 8, and 10 μg of a different lot of rConA (051205). The calibration curve indicated saturation of the pixels above 12 μg loads. Using the 8-12 μg range calibration curve, the 2 μg spike indicated a % recovery of 97.5%. A repeat of the experiment confirmed the findings of the first experiment and resulted in a % recovery of 96.7% at the 2 μg spike level.

Based on the Linearity, Precision, and Accuracy data obtained, the working range for rConA was determined to be 8-12 μg.

(c) Calculation of Purity

To calculate the percentage purity of the eluted recombinant ConA tetramers, the protein gel described above undergoes densitometric analysis. First, the gel was converted to a digitized image by scanning the gel and storing the image in a TIFF or JPG format. Next, the image was digitally corrected to remove background and focus the image using Adobe Photoshop 7.0. The enhanced image was ported to Image Pro 5.0 where values were calculated for each individual protein band within the eluate. These values were then ported to Origin Pro 7.0 at which point the Image Pro 5.0 data was graphically displayed as a series of peaks. To calculate the percentage purity, the ratio of area under a single peak (protein band) versus the sum of the area of all peaks present (total peak area) was calculated. The percentage generated provides a value indicative of the purity of rConA within a single eluate sample. Both purified gConA and mConA, were analyzed. A summary of the results is shown in Table 4

TABLE 4
Summary of ConA Purity Results from SDS-Page

ConA Samples Analyzed by SDS-PAGE

| | mConA | gConA |
|---|---|---|
| Purity % (+/−SD) | 99.6 +/− 0.6 | >99 |
| N | 19 | 1 |
| Analysis Method | Densitometry | Densitometry |
| Antibiotics Used | Kanamycin | Ampicillin |

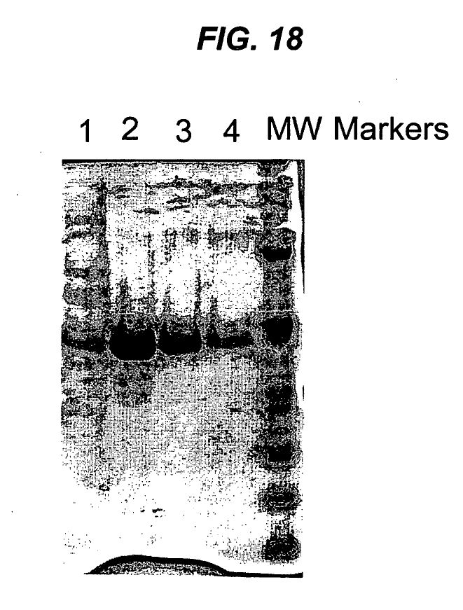

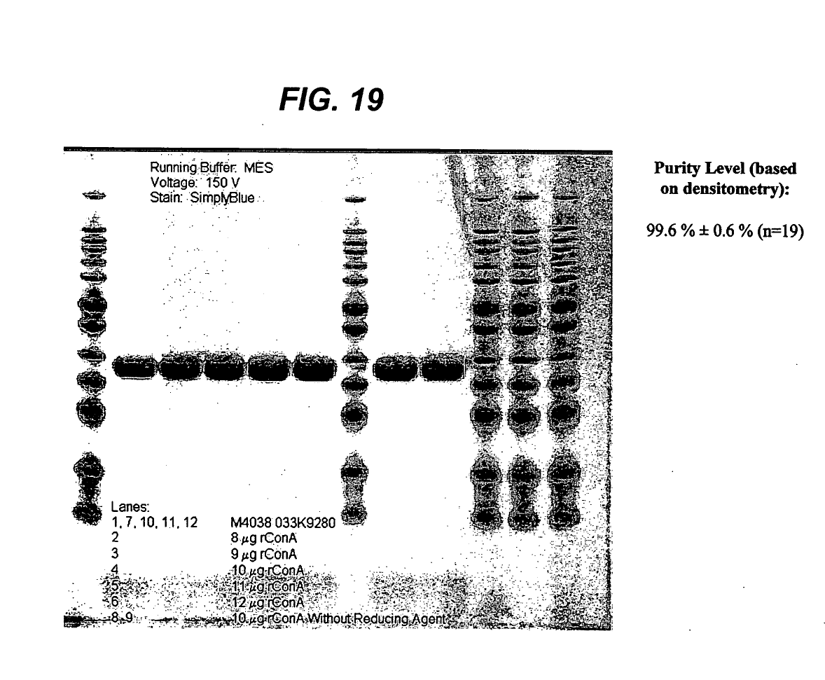

FIG. 18 shows purification of gConA by sephadex column. Three eluates were collected and separated by SDS-PAGE, Lane 1, total refolded. Lane 2, eluate #1. Lane 3, eluate #2. Lane 4, eluate #3. Lane 5, MW markers. The results showed a single band at approximately 26 kDa, which represented purified recombinant gConA. Recombinant mConA was run on SDS-PAGE at different levels of protein from 8-12 μg (FIG. 19).

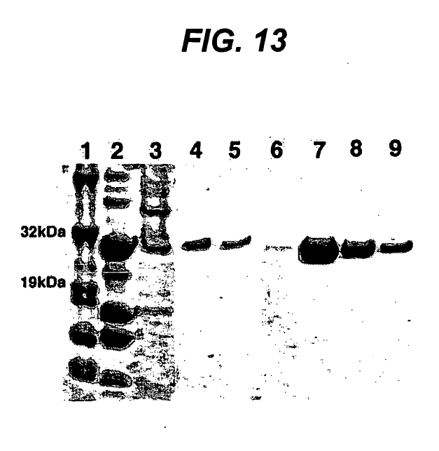

FIG. 13 demonstrates enrichment of the monomeric rConA 26 kDa band during the latter stages of the purification procedure. Lane 1, MW markers. Lane 2, 20 ug nConA marker. Lane 3, denatured inclusion body. Lane 4, renatured supernatant. Lane 5, Sephadex G-75 flowthrough. Lane 6, Sephadex G-75 wash, Lane 7-9, Sephadex G-75 (eluates #1-#3). 20 ul from each stage was run on 10% Bis-Tris acrylamide gel and stained with colloidal blue stain (Simply Blue, Invitrogen).

iii. HPLC

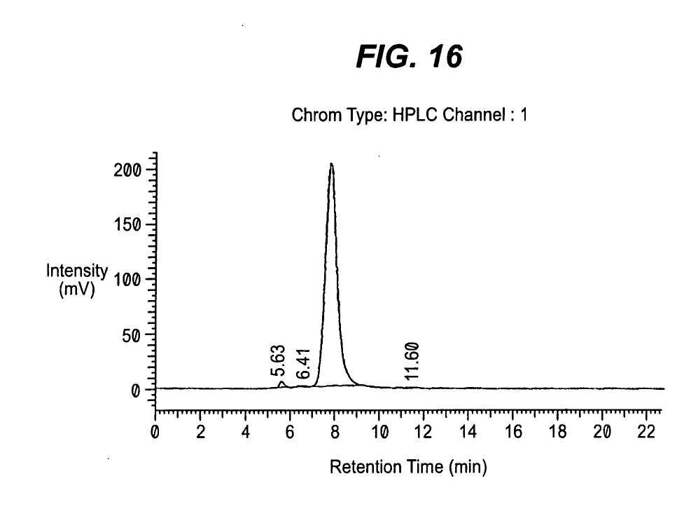

FIG. 16 shows HPLC (SEC-size exclusion chromatography) separation of recombinant gConA. This figure shows purified recombinant gConA to consist of a single primary peak (98.67% by relative peak area integration) eluting at 7.79 minutes (retention time). Automatic peak identification was used and the software identified 4 total peaks in this scan and used each of these peaks to calculate purity by integration of peak areas.

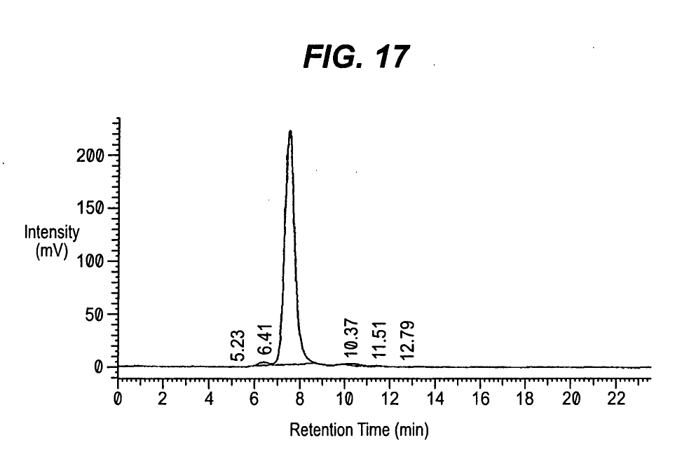

FIG. 17 shows HPLC (SEC-size exclusion chromatography) separation of recombinant mConA. This figure shows purified recombinant mConA to consist of a single primary peak (97.47% by relative peak area integration) eluting at 7.50 minutes (retention time). Automatic peak identification was used and the software identified 6 total peaks in this scan and used each of these peaks to calculate purity by integration of peak areas.

A summary of the HPLC results is set forth in Table 5 below:

TABLE 5
Summary of ConA purity results Analyzed using HPLC

ConA Samples Analyzed by HPLC

| | mConA | gConA |
|---|---|---|
| Purity % (+/−SD) | 98.41 +/− 0.68 | 97.67 +/− 1.06 |
| N | 9 | 2 |
| Analysis Method | Peak integration | Peak integration |
| Antibiotics Used | Kanamycin | Ampicillin |

iv. SEC-MALLS

This method combines separation of proteins using HPLC size-exclusion chromatography (SEC) with simultaneous detection using UV, multi-angle laser light scattering, and refractive index. A Tosoh TSKgel G2000SWXL, 5 μm, 125 ø 7.8 mm×30 cm (Tosoh Product number: 08540), HPLC column was used. The Mobile Phase Buffer System (pH 7.0) consisted of: 400 mM NaCl; 20 mM MOPS; 20 mM a-D Methyl Mannopyranoside; 0.1 mM MnCl2; and 0.1 mM CaCl2. The HPLC was run under the following conditions: Temperature: Room Temperature; Flow Rate: 1 ml/min; ConA Concentration: Between 1 mg/ml and 3 mg/ml; sample Injection size: 100 μl

The following detection equipment was used: A Hitachi L4250 UV-Vis Detector with detection performed at 280 nm; a Wyatt miniDAWN MALS Detector with detection perfomed at 685 nm; and a Wyatt OptiLab rEX Refractive Index Detector with detection at 660 nm or 690 nm

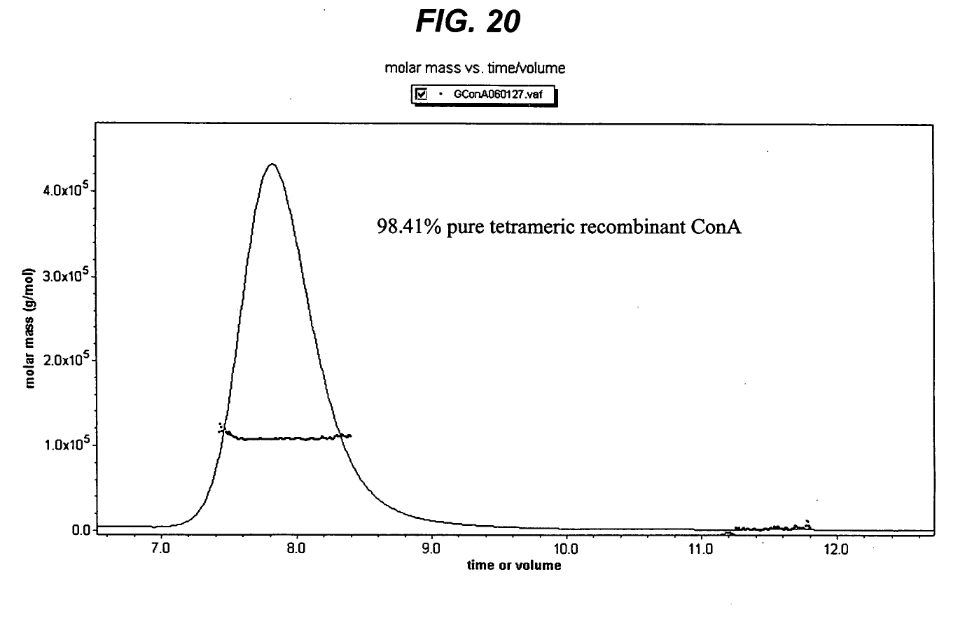

FIG. 20 shows an SEC-MALLS analysis of gConA. The figure depicts both the UV trace (solid line) with the molar mass overlay (symbols) to show both purity of the gConA sample as well as the homogenous distribution of tetramer within the primary peak. Peak integration results: 98.41% by relative peak area integration. The antibiotic used to produce gConA was Ampicillin.

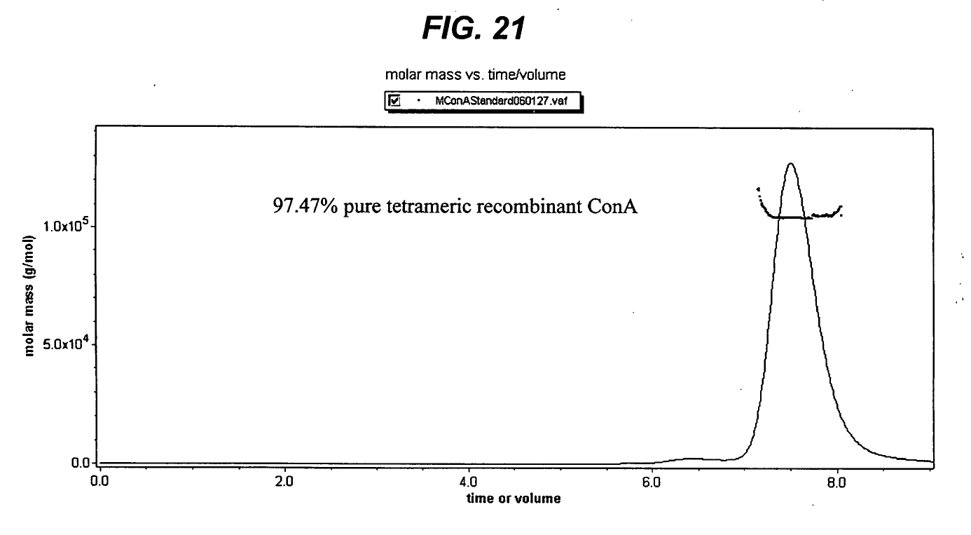

FIG. 21 shows an SEC-MALLS analysis of mConA. The figure depicts both the UV trace (solid line) with the molar mass overlay (symbols) to show both purity of the mConA sample as well as the homogenous distribution of tetramer within the primary peak. Peak integration results: 97.47% by relative peak area integration. The antibiotic used to Produce mConA was Kanamycin.

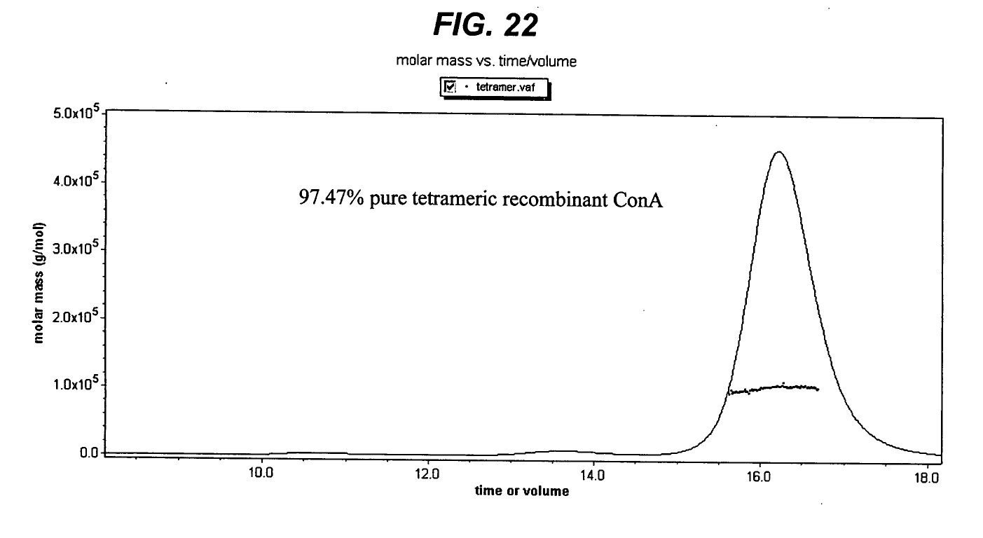

FIG. 22 shows and SEC-MALLS analysis of mConA conducted by Wyatt Industries. The figure depicts the UV trace (solid line) with the molar mass overlay (symbols) to show both purity of the mConA sample as well as the homogenous distribution of tetramer within the primary peak. This experiment was conducted to independently verify results obtained in our laboratory, and was performed by Wyatt Industries (the manufacturer of the MALLS and RI detectors used in these studies). It verifies purity of sample (single primary peak in HPLC) as well as homogenous distribution of molar mass for the tetrameric mConA. The peak integration results indicated a 97.47% purity by relative peak area integration. The antibiotic used to produce the mConA used in this analysis was Kanamycin.

HPLC analysis demonstrated protein purity of recombinant ConA lots, to be greater than 97% pure based on integration of HPLC peaks.

v. Host-Cell Protein (HCP) ELISA

An ELISA system designed to detect HCP contaminants from a number of E. coli, including the strain used in our recombinant protein expression, BL21 was used.

Two hundred (200) μl of control and experimental samples were pipetted into 96-well plate. The plate was covered and incubated on a rotator at 180 rpm for 2 hours at room temperature. Contents of each well were aspirated and washed 3× with 350 μl wash solution. Two hundred (200) μl of anti-E. Coli alkaline phosphatase was added into all wells. The plate was covered and incubated on a rotator for 2 hours at room temperature. The wells were then washed again as described previously. Two hundred (200) μl of substrate was added into all wells. The plate was covered and incubated for 60 minutes. Absorbance was read at 405/492 nm in a plate reader. Results for all ConA sample wells were averaged and an overall value reported and is summarized in Table 6.

TABLE 6
HCP of purified ConA protein

| | ConA Samples Analyzed by HCP ELISA | | |
|---|---|---|---|
| | mConA | mConA | gConA |
| HCP Contaminant | 43.5 +/− 10.4 | 58.21 +/− 16.31 | 91.89 +/− 27.90 |
| (ng HCP/mg | | | |
| purified ConA) | | | |
| (+/−SD) | | | |
| N | 2 | 10 | 5 |
| Antibiotics used | Kanamycin | Ampicillin | Ampicillin |

B. Functional Characterization of ConA

Functional properties of rConA has been characterized by Fluorescence Resonance Energy Transfer (FRET) using a PTI QuantaMaster fluorimeter, and Surface Plasmon Resonance (SPR) using a Biacore 2000. FRET occurs when two dye molecules interact in a distance-dependent fashion. The excitation energy from one dye (the donor) is transferred to a second dye molecule (the acceptor) without photon emission by an electrostatic dipole induced dipole interaction. This transfer of energy results in emission of the acceptor dye, which is one useful way to monitor the interaction of the proteins on which these two dyes reside. SPR measures the change in refractive index at the interface between an immobilized molecule and the solution flowing over this molecule, which results in changes in the angles at which light excitation light is reflected. This change in angle, which can be caused by the association and dissociation of molecules at this interface is proportional to the mass of material bound. This real-time measurement is a rapid way to characterize the functional binding of rConA, and to assess the effect of dye labeling on rConA affinity.

i. Dye Conjugations

Purified rConA can be used in FRET interactions but must first be labeled with a fluorescent dye. rConA was used as both the donor and the acceptor in FRET reactions, using both the Cy (Amersham) and Alexa (Molecular Probes) families of dyes. The conjugation reactions are similar regardless of which dye is used. We have characterized the effect of degree of dye conjugation to rConA (as measured by the molar ratio of dye/protein, or D/P) on FRET performance, and have found that it is preferable to have D/P<1.0. Maximal percent response is usually achieved with D/P between 0.2 and 0.5, and we have targeted our conjugation reactions to achieve this.

Typically, 0.25 mg of dye was used to label 5 mg of rConA. Purified natural ConA as well as rConA from C. ensiformis, and C. gladiata were used in conjugation reactions. In addition, we made a mutant of C. ensiformis, mConA which has been described above.

(a) FRET with Conjugated rConA

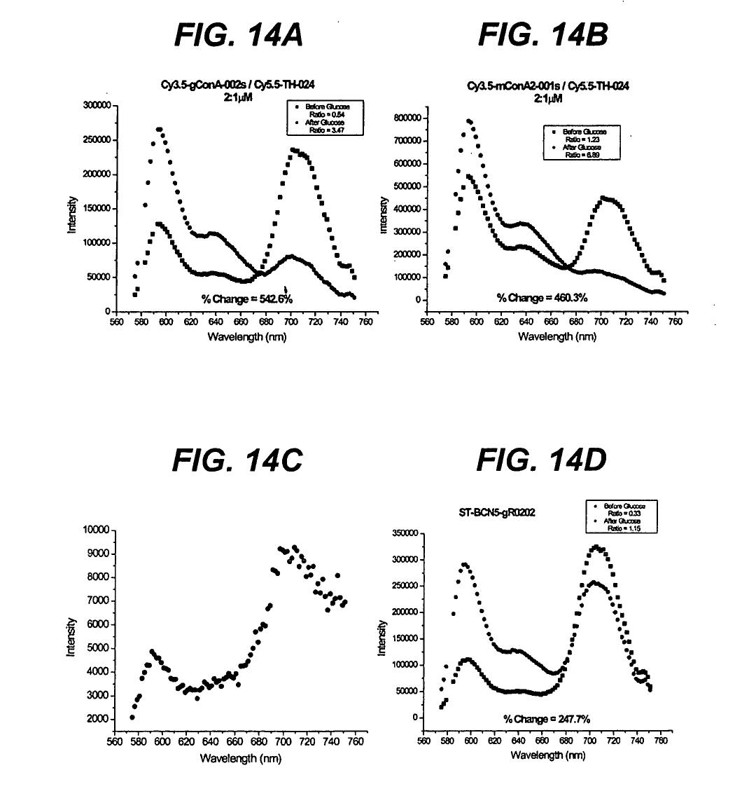

FRET was used to monitor the interaction of dye-labeled rConA with dye and sugar-labeled therapeutic human serum albumin (tHSA). rConA was labeled with the donor, Cy3.5. Therapeutic HSA (tHSA) was labeled with the acceptor, Cy5.5 as well as α-D-glucopyranosylphenyl isothiocyanate (GPITC). Under these conditions, binding of rConA to tHSA when mixed in a ratio of 2 μM rConA to 1 μM tHSA, resulted in efficient FRET. FIG. 14 illustrates the non-radiative transfer of energy from the donor (peak at ˜600 nm) to the acceptor (peak at ˜700 nm) in the presence of 500 mg/dL glucose (circles) and with no glucose (squares). In FIG. 14A, C. gladiata rConA was conjugated to Cy3.5 and mixed with Cy5.5+GPITC conjugated tHSA at a 2 μM to 1 μM ratio. The spectra shows a ˜543% response in the presence of glucose in solution. In FIG. 14B, D58G mutant C. ensiformis rConA was conjugated to Cy3.5 and mixed with Cy5.5+GPITC conjugated tHSA at a 2 μM to 1 μM ratio. The spectra without glucose (squares) and in the presence of 500 mg/dL glucose (circles) show a ˜460% response in solution. FIG. 14C shows a FRET spectra for non-mutated C. ensiformis when D/P was <0.2. This particular conjugate did not result in reversible FRET, which suggests that it bound tightly to tHSA, and was not displaced by the addition of free glucose. Other FRET studies shows that (1) natural ConA purified as described above, resulted in similar FRET performance (data not shown) (2) rConA can labeled with the acceptor instead of the donor, for the Cy family of dyes (data not shown) and (3) recombinant HSA (rHSA) can be used instead of tHSA, but requires a higher degree of GPITC conjugation to achieve similar results (data not shown) and (4) the family of Alexa dyes can be used instead of or in combination with Cy dyes, specific examples are Alexa 568 as the donor and Alexa 647 as the acceptor.

Sensors can be made with conjugated pairs of rConA and tHSA after they have been characterized in solution FRET. Examples of FRET-based sensors are described in U.S. Pat. No. 6,040,194 to Chick et al., which has been incorporated by reference in its entirety. FIG. 14D shows FRET spectra of a sensor made with Cy 3.5 conjugated rConA from C. gladiata and Cy5.5+GPITC conjugated tHSA, when mixed at a final concentration of 3 μM to 1.5 μM ratio. The spectra obtained without glucose (squares) and in the presence of 500 mg/dL glucose (circles) show a ˜248% response when in sensors. Similar spectra were obtained for the D58G mutant C. ensiformis rConA (mConA). Response data was obtained for FRET both in solution and in sensors.

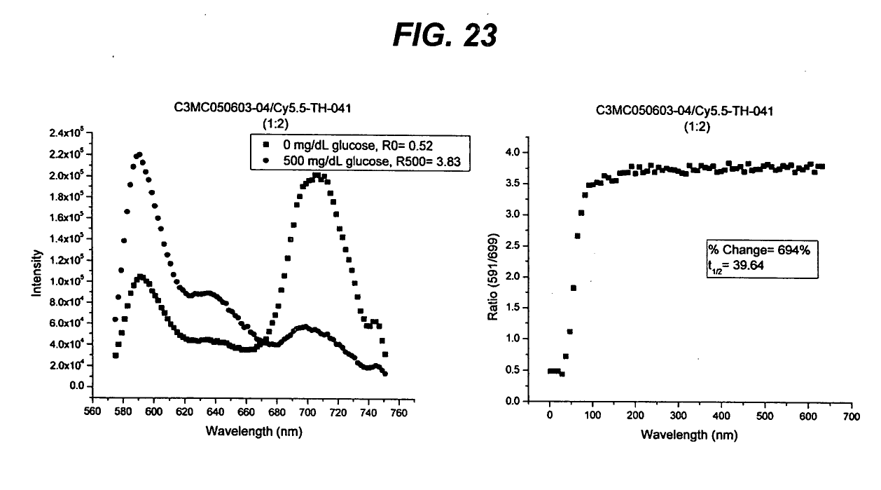

FIG. 23 depicts FRET results for mConA in solution. Conjugates were combined in solution and emission spectra (left panel) obtained both before (black curve) and after (red curve) addition of 500 mg/dL glucose. A time-based scan was acquired which shows the ratio of emission peaks after the addition of 500 mg/dL glucose (right panel). The antibiotic used to prepare this ConA conjugate was ampicillin.

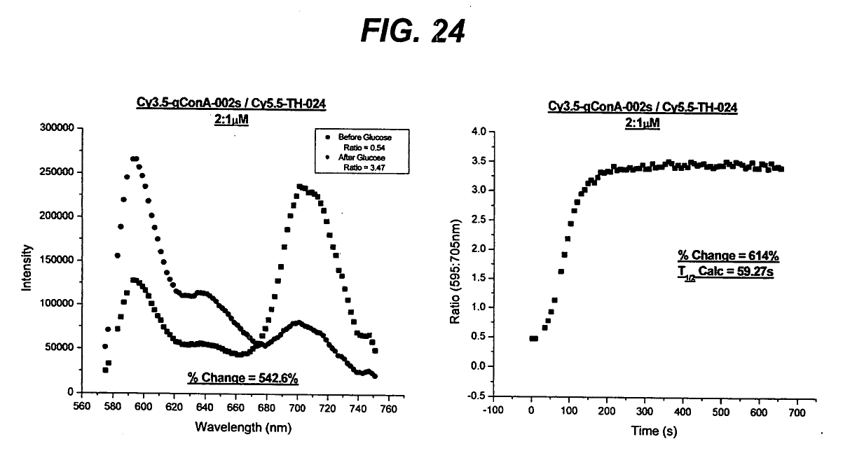

FIG. 24 depicts FRET results for gConA in solution. Conjugates were combined in solution and emission spectra (left panel) obtained both before (black curve) and after (red curve) addition of 500 mg/dL glucose. A time-based scan was acquired which showed the ratio of emission peaks after the addition of 500 mg/dL glucose (right panel). The antibiotic used to prepare this ConA conjugate was ampicillin. Table 7 summarizes the FRET percentage response of conjugates in solution for mConA and gConA.

TABLE 7
Summary of ConA Function Results: FRET Data in Conjugates in
Solution

FRET % Response in Solution

| Sample | Antibiotic Used | Mean +/− SD | N |
|---|---|---|---|
| mConA | Ampicillin | 796 +/− 262 | 36 |
| mConA | Kanamycin | 702 +/− 431 | 29 |
| gConA | Ampicillin | 576 +/− 277 | 10 |

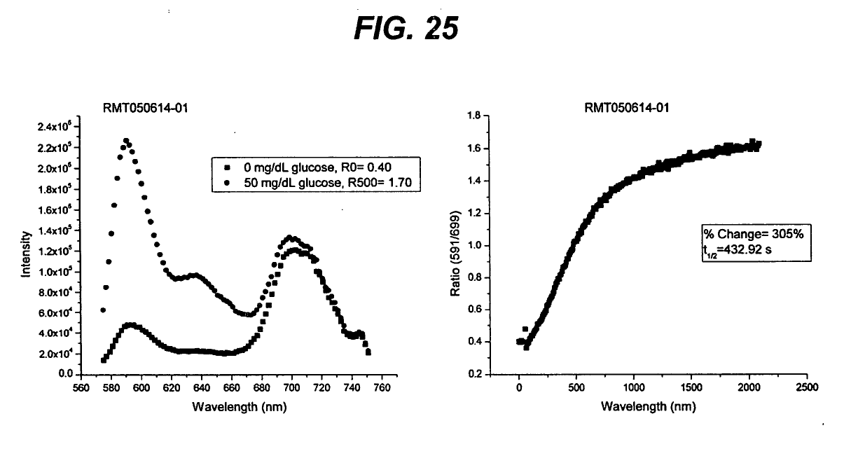

FIG. 25 depicts FRET results for mConA in sensors. Sensors were made using conjugates previously characterized in solution, and emission spectra (left panel) obtained both before (black curve) and after (red curve) addition of 500 mg/dL glucose. The spectra shows a 305% response in the presence of glucose when in sensors. A time-based scan was also acquired which showed the ratio of emission peaks after the addition of 500 mg/dL glucose (right panel). The antibiotic used to prepare this ConA conjugate was ampicillin.

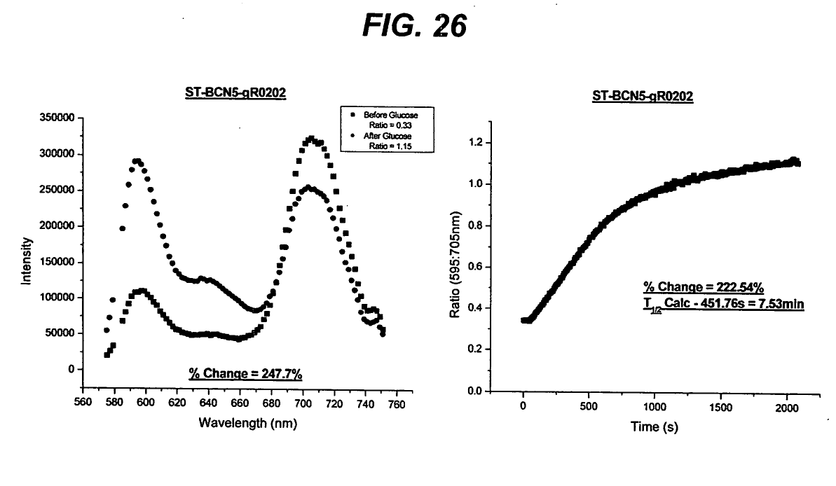

FIG. 26 depicts FRET results for gConA in sensors. Sensors were made using conjugates previously characterized in solution, and emission spectra (left panel) obtained both before (black curve) and after (red curve) addition of 500 mg/dL glucose. The spectra show a 223% response in the presence of glucose when in sensors. A time-based scan was acquired which shows the ratio of emission peaks after the addition of 500 mg/dL glucose (right panel). The antibiotic used to prepare this ConA conjugate was ampicillin.

TABLE 8
Summary of ConA Function
Results: FRET Data in Sensors

FRET % Response in Sensors

| Sample | Antibiotic Used | Mean +/− SD | N |
|---|---|---|---|
| mConA | Ampicillin | 407 +/− 146 | 34 |
| mConA | Kanamycin | 283 +/− 130 | 27 |
| gConA | Ampicillin | 287 +/− 108 | 6 |

(b) Affinity of ConA Using Surface Plasmon Resonance

Natural ConA was characterized by Surface Plasmon Resonance (SPR), using the Biacore 2000 with immobilized GPITC-conjugated tHSA. tHSA alone (not GPITC conjugated), as well as differing levels of GPITC-conjugated tHSA were immobilized on dextran-derivatized chips, and solutions of various ConA concentrations analyzed by flow over this chip.

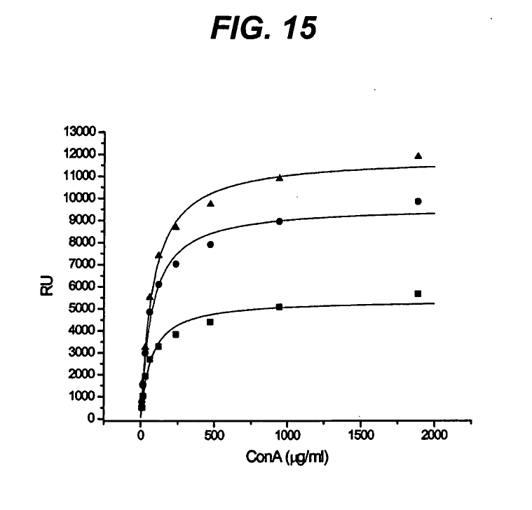

FIG. 15 shows increased binding of ConA at three different level of immobilized GPITC-tHSA, when ConA solutions of increasing concentration were used. Increasing Response Units reflect greater ConA binding at increasing GPITC-tHSA levels, resulting in greater Bmax values. The Bmax values for the three levels of immobilized GPITC-tHSA were 5400, 9600 and 12000 RU respectively. The affinity of ConA binding to GPITC-tHSA was not expected to change with increasing immobilized GPITC-tHSA. The affinity of ConA binding for these levels of tHSA are 67, 70 and 77 μg/ml respectively. Table 9, shown below summarizes the data providing affinity (KD) values for gConA and mConA. Both gConA and mConA were produced using ampicillin.

TABLE 9
Summary of Biacore Data

| Sample | KD₁ (μg/ml) | KD₂ (μg/ml) | BMAX₁ | BMAX₂ |
|---|---|---|---|---|
| gConA | 1.89 | 41.32 | 25% | 75% |
| mConA | 0.66 | 49.49 | 55% | 45% |

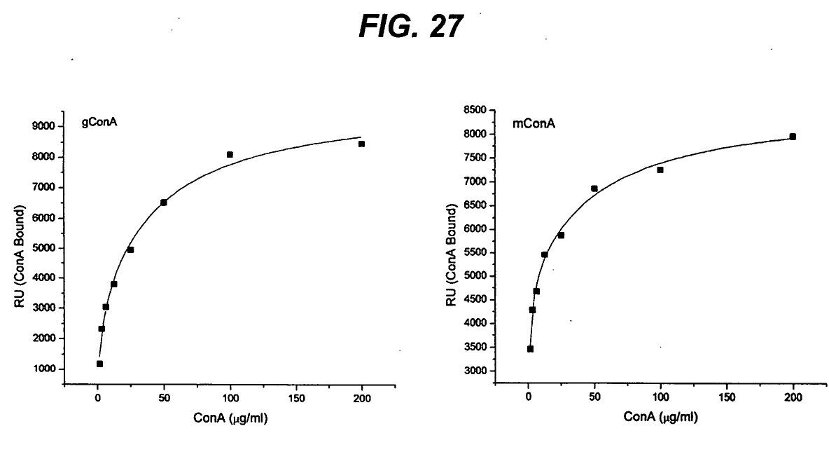

FIG. 27 depicts Binding of gConA (left panel) and mConA (right panel) to GPITC-tHSA. GPITC-conjugated tHSA was immobilized on dextran-derivatized chips. Solutions with increasing ConA concentration (0.78 to 200 μg/ml) were exposed to immobilized GPITC-tHSA at a flow rate of 101 μl/minute for a total contact time of 10 minutes. The data was fit to a two component saturaticin binding isotherm and parameter estimates for Bmax and Kd determined as shown Table 9.

### REFERENCES

The references cited herein, to the extent that they provide exemplary procedural or other details supplementary to those set forth herein, are specifically incorporated by reference.
Abe, Y., M. Iwabuchi, and S. I. Ishii, Multiple forms in the subunit structure of concanavalin A. Biochem Biophys Res Commun, 1971. 45(5): p. 1271-8.
Auer, H. E. and T. Schilz, Denaturation of concanavalin A by urea at acid pH. Int J Pept Protein Res, 1984. 24(6): p. 569-79.
Auer, H. E. and T. Schilz, pH-dependent changes in properties of concanavalin A in the acid pH range. Int J Pept Protein Res, 1984. 24(5): p. 462-71.
Brennan, T. V. and S. Clarke, Mechanism of cleavage at Asn 148 during the maturation of jack bean concanavalin A. Biochem Biophys Res Commun, 1993. 193(3): p. 1031-7.
Carrington, D. M., A. Auffret, and D. E. Hanke, Polypeptide ligation occurs during post-translational modification of concanavalin A. Nature, 1985. 313(5997): p. 64-7.
Clark, E. D. B., Refolding of recombinant proteins. Curr Opin Biotechnol, 1998. 9(2): p. 157-63.
Cunningham, B. A., et al., Isolation and proteolytic cleavage of the intact subunit of concanavalin A. Biochemistry, 1972. 11(17): p. 3233-9.
Haugland, R. P., The Handbook—A Guide to Fluorescent Probes and Labeling Technologies, 10^{th} Ed., 2005 Invitrogen, Eugene Oreg.
Hermanson, G. T., Bioconjugate Techniques, 1996, Academic Press Inc, San Diego.
Huet, M., Factors affecting the molecular structure and the agglutinating ability of concanavalin A and other lectins. Eur J Biochem, 1975. 59(2): p. 627-32.
Lefebvre, B., P. Formstecher, and P. Lefebvre, Improvement of the gene splicing overlap (SOE) method. Biotechniques, 1995. 19(2): p. 186-8.
Liener, I. E., N. Sharon, and I. J. Goldstein, eds. The Lectins: Properties, Functions, and Applications in Biology and Medicine. Molecular Biology: An International Series of Mongraphs and Textbooks, ed. B. Horecker, et al. 1986, Academic Press, Inc.: Orlando. 600.
Markossian, K. A. and B. I. Kurganov, Protein folding, misfolding, and aggregation. Formation of inclusion bodies and aggresomes. Biochemistry (Mosc), 2004. 69(9): p. 971-84.
Min, W., Emulation of the Post-translational Processing of Concanavalin A by Recombinant DNA Manipulations, in School of Biological Sciences. 1992, University College of Swansea: Swansea. p. 255.
Rentier-Delrue, F., D. Swennen, and J. Martial, pIN-III-ompA secretion vectors: modification of the ompA signal peptide sequence for easier insert cloning. Nucleic Acids Res, 1988. 16(17): p. 8726.
Sambrook et al., Molecular Cloning a Laboratory Manual, 3d Ed., 2001, Cold Spring Harbor Press.
Sheldon, P. S. and D. J. Bowles, The glycoprotein precursor of concanavalin A is converted to an active lectin by deglycosylation. Embo J, 1992. 11(4): p. 1297-301.
Sheldon, P. S., J. N. Keen, and D. J. Bowles, Post-translational peptide bond formation during concanavalin A processing in vitro. Biochem J, 1996. 320 (Pt 3): p. 865-70.
Tsumoto, K., et al., Role of arginine in protein refolding, solubilization, and purification. Biotechnol Prog, 2004. 20(5): p. 1301-8.
Villayerde, A. and M. M. Carrio, Protein aggregation in recombinant bacteria: biological role of inclusion bodies. Biotechnol Lett, 2003. 25(17): p. 1385-95.
Warrens, A. N., M. D. Jones, and R. I. Lechler, Splicing by overlap extension by PCR using asymmetric amplification: an improved technique for the generation of hybrid proteins of immunological interest. Gene, 1997. 186(1): p. 29-35.

What is claimed is:

1. A composition comprising a substantially purified lectin polypeptide wherein the composition is at least 95% pure.

2. A composition comprising a substantially purified lectin polypeptide wherein the lectin comprises greater than 95% by weight of the total protein of the composition.

3. A composition comprising a substantially purified lectin polypeptide wherein the composition has a purity of greater than 95% as determined by relative peak area integration.

4. The composition of claim 2 wherein the composition has a purity of greater than 97% by relative peak integration.

5. The composition of claims 1 or 2 wherein the lectin polypeptide comprises recombinant Concanavalin A.

6. The composition of claims 1 or 2 wherein the lectin is a tetramer.

7. The composition of claims 1 or 2 wherein the lectin is a dimer.

8. The composition of claims 1 or 2 wherein the lectin is a monomer.

9. The composition of claims 1 or 2 wherein the lectin polypeptide comprises a mutant recombinant Concanavalin A.

10. The composition of claims 1 and 2 wherein the lectin polypeptide comprises a tetramer of the polypeptide of SEQ ID NO: 15.

11. A method of producing a recombinant lectin of interest comprising inducing expression of said lectin in a bacterial cell culture.

12. The method of claim 11 further comprising:
(a) lysing the cells of the bacterial culture to produce an inclusion body fraction;
(b) purifying the inclusion body fraction;
(c) solubilizing the inclusion bodies in the inclusion body fraction so that the lectin of interest is present in solution;
(d) denaturing the lectin of interest;
(e) allowing the lectin of interest to refold in solution; and
(f) purifying the solution.

13. The method of claim 11 wherein the cells of the bacterial culture have been transformed by a vector comprising a kanamycin resistance gene.

14. The method of claim 11 wherein the transformed bacterial cell culture is induced with IPTG in the absence of kanamycin.

15. The method of claim 12 wherein denaturing the lectin of interest occurs at a pH of less than 5.

16. The method of claim 12 wherein the solution is purified by affinity chromatography.

17. The method of claim 12 wherein the solution is purified by size-exclusion chromatography.

18. The method of claim 16 wherein the solution is purified by size-exclusion chromatography.

19. The method of claim 11 wherein the lectin is a member of a family of proteins that specifically bind at least one of glucose and mannose.

20. The method of claim 19 wherein the lectin is a Concanavalin A.

21. The method of claim 20 wherein the lectin comprises the polypeptide of SEQ ID No:15.

22. A method of purifying a lectin comprising:
adding a denaturing, chaotropic agent to a solution of lectin having a pH less than 5, and
subjecting said solution to size exclusion chromatography.

23. The method of claim 22 wherein the lectin is a Concanavalin A.

24. A composition comprising a substantially purified lectin having less than about 150 ng of Host Cell Protein (HCP) per mg of purified lectin.

25. The lectin of claim 24 comprising a Concanavalin A.

26. The lectin of claim 24 comprising a mutant Concanavalin A.

27. The lectin of claim 24 comprising the polypeptide of SEQ ID NO:15.

28. An isolated nucleic acid sequence encoding a mutant form of a natural Concanavalin A.

29. The isolated nucleic acid of claim 28 comprising SEQ ID No. 16.

30. The isolated nucleic acid of claim 28 operatively linked to a promoter.

31. A host cell that contains the nucleic acid of claim 28 and expresses the encoded protein.

32. A polypeptide coded for by the nucleic acid sequence of claim 28.

33. The polypeptide of claim 32 comprising SEQ ID No. 15.

34. A method of producing a Concanavalin A exhibiting reduced precipitation during purification comprising performing a mutation to the nucleic acid sequence of a Concanavalin A wherein the mutation encodes for an amino acid change, the amino acid change converting an acidic amino acid site to a neutral amino acid.

35. A vector comprising an inducible promoter, a kanamycin resistance gene and a nucleic acid sequence encoding for a form of Concanavalin A.

36. The vector of claim 35 wherein the nucleic acid sequence is comprised of the sequence of SEQ ID NO:16.

37. A sensor comprising a mutant form of Concanavalin A.

38. The sensor of claim 37 wherein the mutant form of Concanavalin A has at least one mutation encoding for an amino acid change, the amino acid change converting an acidic amino acid site to a neutral amino acid.

39. The sensor of claim 38 wherein the mutant Concanavalin A comprises the polypeptide of SEQ ID NO:15.

40. The sensor of claim 37 further comprising:
(a) a donor; and
(b) an acceptor,
wherein the mutant Concanavalin A is labeled with at least one of the donor and the acceptor.

41. The sensor of claim 40 further comprising a fluorescence acceptor conjugated to a glycosylated substrate.

42. The sensor of claim 40 further comprising a fluorescent donor conjugated to a glycosylated substrate.

43. The sensor of claim 40 wherein the mutant Concanavalin A comprises the polypeptide of SEQ ID NO:15.


SEQUENCE LISTING (20 sequences)

SEQ ID NO: 1 - DNA, 714 bp, Artificial
  Chemically synthesized
gccgatactattgttgctgttgaattggatacttatcccaatactgatattggagatccaagttatccacacatcggtatcgatattaaatctgttcgctccaagaagaccgcaaagtggaacatgcaaaatgggaaggtaggcactgcacacatcatctataactctgttgataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagcaattcaacacatgagacaaatgcactccatttcatgttcaaccaatttagcaaagatcagaaggatttgatccttcaaggtgacgccacaacaggaacagagggtaacttgagactcacaagggtgtcaagtaatgggagtccacagggaagcagtgtgggccgggctttgttctatgccccagtccacatttgggaaagttctgctgtggtggccagctttgaagctacctttacatttctcataaaatcacccgactctcacccagctgatggaattgccttcttcatttcaaatattgacagttcaatccctagtggttccactggaaggctcttgggactcttccctgatgcaaattga

SEQ ID NO: 2 - DNA, 714 bp, Artificial
  Chemically synthesized
gccgatactattgttgctgttgaattggatacctatcccaatactgatattggagatccaaattatccacacatcggtatcgatattaaatctgttcgctccaagaagaccgcaaagtggaacatgcaaaatggaaaggtaggcactgcacacatcatctataactctgttggtaagagactaagtgctgttgtttcttatcctaacggtgactctgccactgtctcttacgacgttgacctcgacaatgtccttcctgaatgggttagagttggcctttctgcttcaaccggactttacaaagaaaccaataccattctctcatggtcttttacttctaagttgaagagcaattcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttgatgctacctttacatttctcataaaatcacccgactctcacccagctgatggaattgccttcttcatttcaaatattgacagttccatccctagtggttccactggaaggctccttggactcttccctgatgcaaattga

SEQ ID NO: 3 - protein, 237 aa, Artificial
  Chemically synthesized
ADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNSTHETNALHFMFNQFSKDQKDLILQGDATTGTEGNLRLTRVSSNGSPQGSSVGRALFYAPVHIWESSAVVASFEATFTFLIKSPDSHPADGIAFFISNIDSSIPSGSTGRLLGLFPDAN

SEQ ID NO: 4 - protein, 237 aa, Artificial
  Chemically synthesized
ADTIVAVELDTYPNTDIGDPNYPHIGIDIKSVRSKKTAKWNMQNGKVGTAHIIYNSVGKRLSAVVSYPNGDSATVSYDVDLDNVLPEWVRVGLSASTGLYKETNTILSWSFTSKLKSNSTHETNALHFMFNQFSKDQKDLILQGDATTGTDGNLELTRVSSNGSPQGSSVGRALFYAPVHIWESSAVVASFDATFTFLIKSPDSHPADGIAFFISNIDSSIPSGSTGRLLGLFPDAN

SEQ ID NO: 5 - protein, 290 aa, Artificial
  Chemically synthesized
MAISKKSSLFLPIFTFITMFLMVVNKVSSSTHETNALHFMFNQFSKDQKDLILQGDATTGTEGNLRLTRVSSNGSPQGSSVGRALFYAPVHIWESSAVVASFEATFTFLIKSPDSHPADGIAFFISNIDSSIPSGSTGRLLGLEPDANVIRNSTTIDFNAAYNADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNEIPDIATVV

SEQ ID NO: 6 - DNA, 20 bp, Artificial
  Chemically synthesized
attgtagcaagcagcactac

SEQ ID NO: 7 - DNA, 21 bp, Artificial
  Chemically synthesized
tagcaagcagcactactagtg

SEQ ID NO: 8 - DNA, 20 bp, Artificial
  Chemically synthesized
gcaagcagcactactagtga

SEQ ID NO: 9 - DNA, 24 bp, Artificial
  Chemically synthesized
gagattattatggtacatggatga

SEQ ID NO: 10 - DNA, 873 bp, Artificial
  Chemically synthesized
atggccatctcaaagaaatcctccctgttccttcctatatttacgttcatcaccatgttcctaatggtagtgaacaaggtgagttcatcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttgaagctacctttacatttctcataaaatcacccgactctcacccagctgatggaattgccttcttcatttcaaatattgacagttccatccctagtggttccactggaaggctccttggactcttccctgatgcaaatgttatcagaaattccactactattgatttcaacgctgcttacaatgccgatactattgttgctgttgaattggatacctatcccaatactgatattggagatccaagttatccacacatcggtatcgatattaaatctgttcgctccaagaagaccgcaaagtggaacatgcaaaatggaaaggtaggcactgcacacatcatctataactctgttgataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagcaatgagatcccggacattgctaccgtggtttga

SEQ ID NO: 11 - DNA, 30 bp, Artificial
  Chemically synthesized
gccgatactattgttgctgttgaattggat

SEQ ID NO: 12 - DNA, 60 bp, Artificial
  Chemically synthesized
gaaatggagtgcatttgtctcatgtgttgaattgctcttcaacttagaagtaaaagacca

SEQ ID NO: 13 - DNA, 60 bp, Artificial
  Chemically synthesized
tggtcttttacttctaagttgaagagcaattcaacacatgagacaaatgcactccatttc

SEQ ID NO: 14 - DNA, 33 bp, Artificial
  Chemically synthesized
tcaatttgcatcagggaagagtccaaggagcct

SEQ ID NO: 15 - protein, 237 aa, Artificial
  Chemically synthesized
ADTIVAVELDTYPNTDIGDPSYPHIGIDIKSVRSKKTAKWNMQNGKVGTAHIIYNSVGKRLSAVVSYPNADSATVSYDVDLDNVLPEWVRVGLSASTGLYKETNTILSWSFTSKLKSNSTHETNALHFMFNQFSKDQKDLILQGDATTGTEGNLRLTRVSSNGSPQGSSVGRALFYAPVHIWESSAVVASFEATFTFLIKSPDSHPADGIAFFISNIDSSIPSGSTGRLLGLFPDAN

SEQ ID NO: 16 - DNA, 714 bp, Artificial
  Chemically synthesized
gccgatactattgttgctgttgaattggatacttatcccaatactgatattggagatccaagttatccacacatcggtatcgatattaaatctgttcgctccaagaagaccgcaaagtggaacatgcaaaatgggaaggtaggcactgcacacatcatctataactctgttggtaagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagcaattcaacacatgagacaaatgcactccatttcatgttcaaccaatttagcaaagatcagaaggatttgatccttcaaggtgacgccacaacaggaacagagggtaacttgagactcacaagggtgtcaagtaatgggagtccacagggaagcagtgtgggccgggctttgttctatgccccagtccacatttgggaaagttctgctgtggtggccagctttgaagctacctttacatttctcataaaatcacccgactctcacccagctgatggaattgccttcttcatttcaaatattgacagttcaatccctagtggttccactggaaggctcttgggactcttccctgatgcaaattga

SEQ ID NO: 17 - DNA, 15 bp, Artificial
  Chemically synthesized
gttgataagagacta

SEQ ID NO: 18 - DNA, 15 bp, Artificial
  Chemically synthesized
gttggtaagagacta

SEQ ID NO: 19 - protein, 5 aa, Artificial
  Chemically synthesized
VDKRL

SEQ ID NO: 20 - protein, 5 aa, Artificial
  Chemically synthesized
VGKRL